(19) United States
(12) Patent Application Publication
(10) Pub. No.: US 2013/0109735 A1
(43) Pub. Date: May 2, 2013

(54) SALACINOL AND PONKORANOL HOMOLOGUES, DERIVATIVES THEREOF, AND METHODS OF SYNTHESIZING SAME

(71) Applicants: Brian Mario Pinto, Coquitlam (CA); Sankar Mohan, Burnaby (CA); Ravindranath Nasi, Ottawa (CA); Jayakanthan Kumarasamy, Toronto (CA); Razieh Eskarandi, Burnaby (CA)

(72) Inventors: Brian Mario Pinto, Coquitlam (CA); Sankar Mohan, Burnaby (CA); Ravindranath Nasi, Ottawa (CA); Jayakanthan Kumarasamy, Toronto (CA); Razieh Eskarandi, Burnaby (CA)

(73) Assignee: SIMON FRASER UNIVERSITY, Burnaby, BC (CA)

(21) Appl. No.: 13/513,142

(22) PCT Filed: Dec. 1, 2010
(86) PCT No.: PCT/CA10/01921

§ 371 Date: Jul. 18, 2012

Related U.S. Application Data
(60) Provisional application No. 61/265,695, filed on Dec. 1, 2009.

(51) Int. Cl. C07D 333/46; C07D 207/12; C07D 493/04; C07D 345/00

(52) U.S. Cl. CPC C07D 333/46; C07D 345/00; C07D 207/12; C07D 493/04
USPC 514/425; 549/66; 514/445; 540/1; 514/183; 548/556; 549/351

(57) ABSTRACT

Salacinol and ponkoranol homologues, derivatives thereof and methods of synthesizing and using said homologies and derivatives. The derivatives include stereoisomers, de-O-sulfonated compounds and congeners of the naturally occurring homologues. Some of the derivatives exhibit enhanced glucosidase inhibitory bioactivity in comparison to the naturally occurring compounds which have been isolated from Salacia reticulata.

### REFERENCE TO RELATED APPLICATIONS

This application claims priority to, and the benefit under 35 U.S.C. §119 of, U.S. provisional patent application No. 61/265,695 filed 1 Dec. 2009 and entitled SALACINOL HOMOLOGUES, DERIVATIVES THEREOF AND METHODS OF SYNTHESIZING SAME, the entirety of which is hereby incorporated by reference.

### FIELD OF THE INVENTION

This application relates to salacinol and ponkoranol homologues, derivatives thereof and methods of synthesizing and using same.

### BACKGROUND OF THE INVENTION

Glycosidases are enzymes that are involved in the catabolism of glycoproteins and glycoconjugates and the biosynthesis of oligosaccharides. Disruption in regulation of glycosidases can lead to diseases.^{1, 2} Over the years, glycosidase inhibitors have received considerable attention in the field of chemical and medicinal research³ because of their effects on quality control, maturation, transport, secretion of glycoproteins, and cell-cell or cell-virus recognition processes. This principle has potential for many therapeutic applications, such as in the treatment of diabetes, cancer and other viral infections.¹

Bioactive components isolated from medicinal plants that are used in traditional medicine or folk medicine often provide the lead structures for modern drug-discovery programs. For example, the large woody climbing plant Salacia reticulata, known as Kothalahimbutu in Singhalese, is used in traditional medicine in Sri Lanka and Southern India for treatment of type 2 diabetes.^{4, 5} A person suffering from diabetes was advised to drink water stored overnight in a mug carved from Kothalahimbutu wood.⁶ Several potent glucosidase inhibitors have been isolated from the water soluble fraction of this plant extract and also other plants that belong to the Salacia genus such as Salacia chinensis, Salacia prinoides, and Salacia oblonga which explain, at least in part, the antidiabetic property of the aqueous extract of this plant.⁷⁻⁹ All these compounds share a common structural motif that comprises a 1,4-anhydro-4-thio-D-arabinitol and a polyhydroxylated side chain. So far, five components have been isolated, namely salaprinol 1,⁹ salacinol 2,⁷ ponkoranol 3,⁹ kotalanol 4,⁸ and de-O-sulfonated kotalanol 5¹⁰ (Chart 1, below). The absolute stereostructure for these compounds, except salacinol, was not determined at the time of isolation, but synthetic work has led to their stereochemical structure elucidation.^{11, 12}

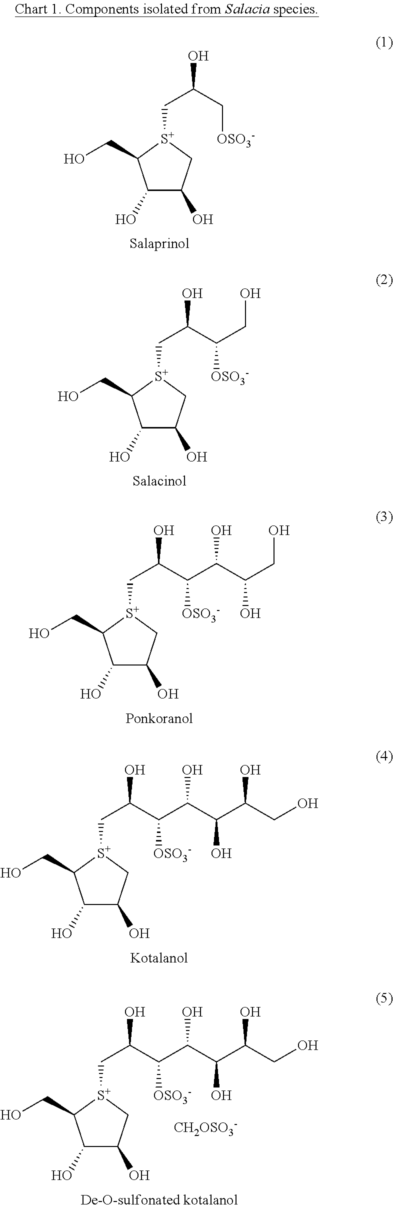

This application relates to higher homologues of salacinol 2 and ponkoronal 3, derivatives thereof and methods of synthesizing same. The derivatives include stereoisomers, de-O-sulfonated compounds and congeners of the naturally occurring homologues. Some of the derivatives exhibit enhanced glucosidase inhibitory bioactivity in comparison to the naturally occurring Salacia isolates.

### SUMMARY

The following embodiments and aspects thereof are described and illustrated in conjunction with systems, tools and methods which are meant to be exemplary and illustrative, not limiting in scope.

In embodiments of the invention, compounds having the structures I, II, or III are provided:

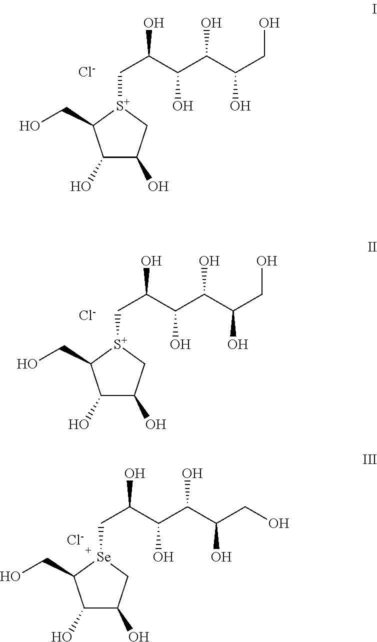

In embodiments of the invention, compounds having the structures IV, V, VI, VII, VIII, IX, X, XI or XII are provided.

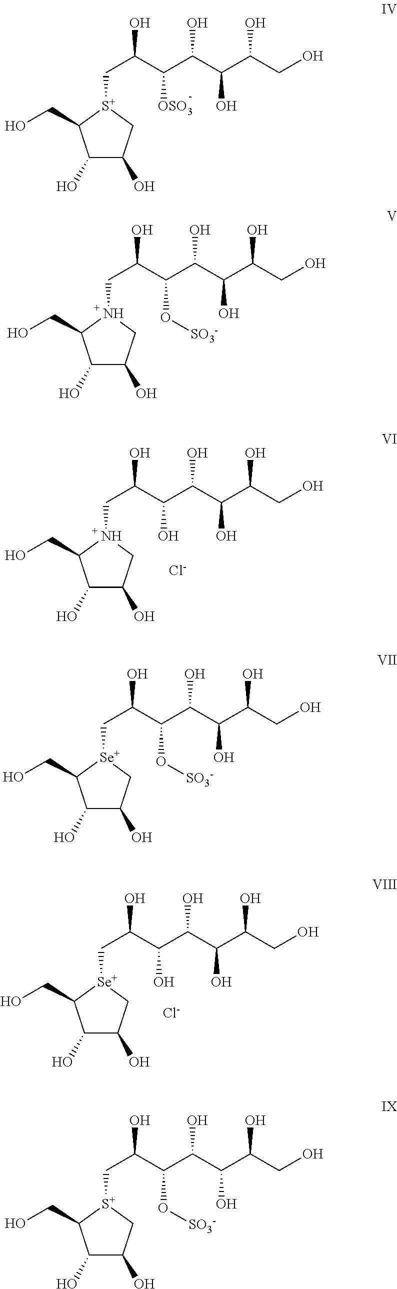

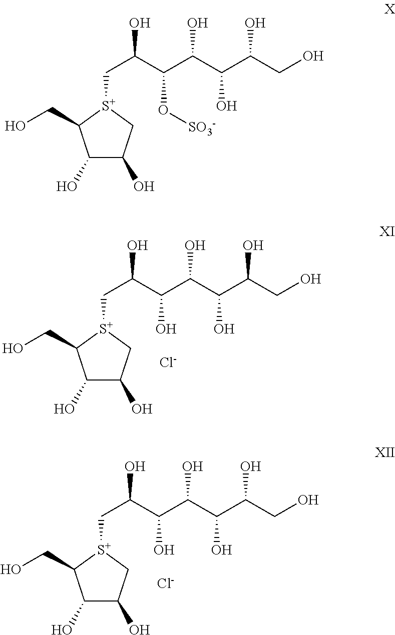

Methods for synthesizing kotalanol and ponkoranol, as well as stereoisomers and analogues thereof, are also provided.

In some embodiments, the compounds are used for the inhibition of glycosidases, such as intestinal glycosidases. In one embodiment, a method of treating diabetes by administering to an affected patient a therapeutically effective amount of the compound is provided.

In addition to the exemplary aspects and embodiments described above, further aspects and embodiments will become apparent by reference to the following detailed descriptions.

### DETAILED DESCRIPTION

Throughout the following description specific details are set forth in order to provide a more thorough understanding of the invention. However, the invention may be practiced without these particulars. In other instances, well known elements have not been shown or described in detail to avoid unnecessarily obscuring the present invention. Accordingly, the specification and drawings are to be regarded in an illustrative, rather than a restrictive, sense.

This application relates to salacinol and ponkoranol homologues, derivatives thereof and methods of synthesizing same.

### 1.0 Synthesis of Kotalanol 5 and its Stereoisomer 6

The inventors have previously described methods of synthesizing kotalanol 4 and de-O-sulfonated kotalanol 5. The present application describes an alternative synthesis of kotalanol 4 as well as a general synthetic route to the kotalanol stereoisomer 6 shown in Chart 2 below.

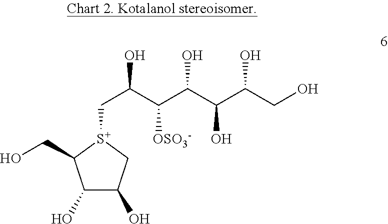

The inventors' first attempted synthesis of kotalanol and its isomer employed the reaction of the cyclic sulfates 8 and 9 in a coupling reaction (Scheme 1).¹² However, attempts to remove the methylene acetal in the coupled products required forcing conditions and resulted in de-O-sulfonation (Scheme 1).¹² The inventors have also reported a successful synthesis of kotalanol using a cyclic sulfate derived from a naturally occurring heptitol, perseitol (Scheme 2).¹²

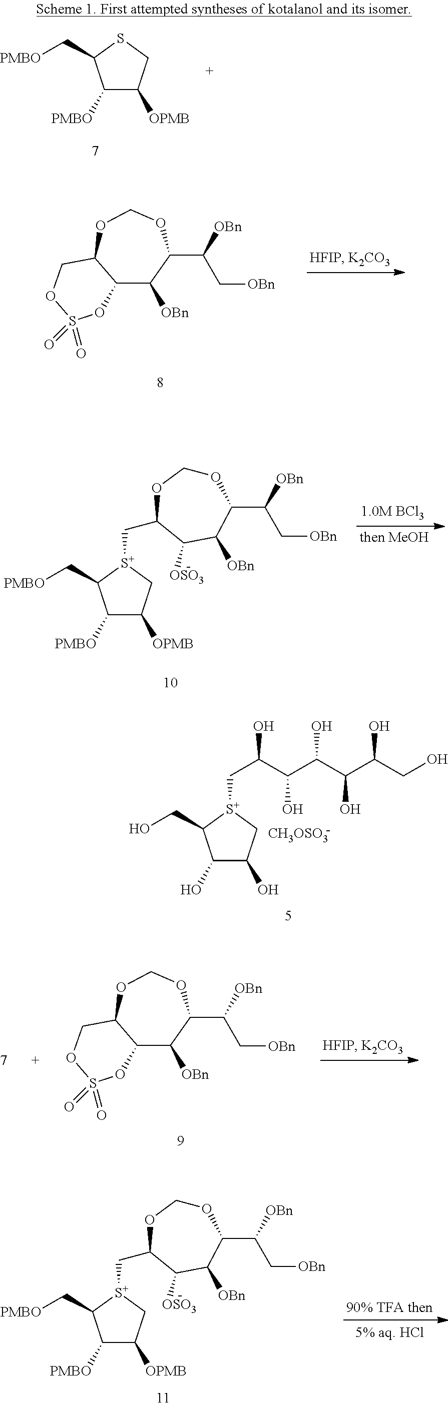

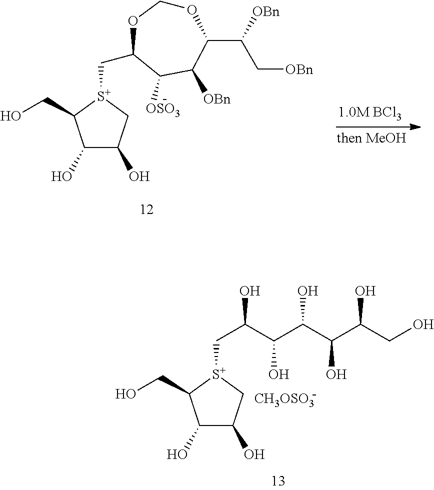

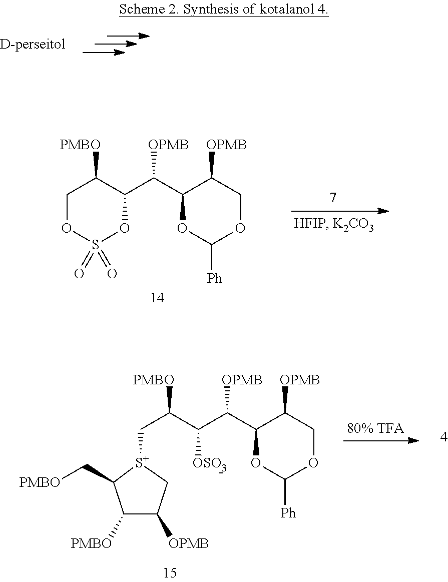

It was of interest to develop a synthesis of the isomer of kotalanol 6 in view of the fact that the isomer of de-O-sulfonated kotalanol 13 was just as active an inhibitor as de-O-sulfonated kotalanol 5 itself against a key intestinal enzyme, human maltase glucoamylase.¹²

In one embodiment the inventors chose to replace the methylene acetal group of compounds 8 or 9 with an isopropylidene acetal (compound 16) to ensure not only its facile removal after the coupling reaction but also to maintain some rigidity in the cyclic sulfate. The inventors chose also to replace the benzyl ethers with methoxymethyl (MOM) ethers, because the latter can survive the hydrogenolysis conditions required for removal of the benzylidene acetal. The cyclic sulfate 16 could be synthesized from D-mannitol as shown in the retrosynthetic analysis (Scheme 3).

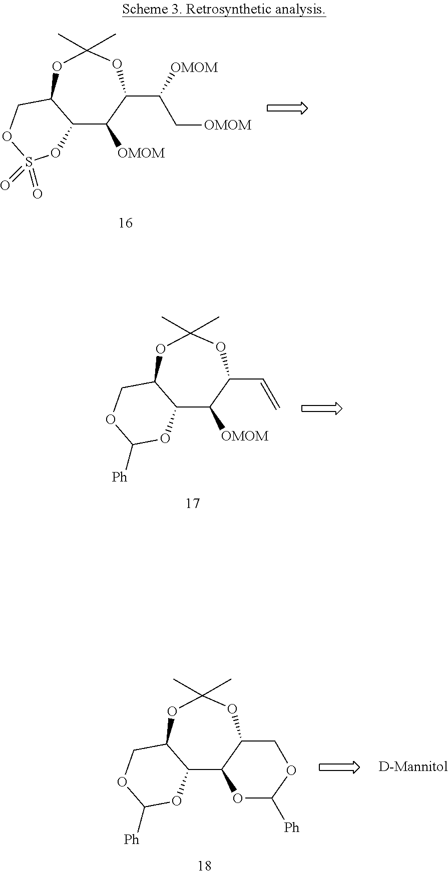

The D-mannitol-derived diol 19,¹³ was protected as the acetonide to give the C₂-symmetric compound 18 in 73% yield. Mild hydrolysis of this compound using catalytic PTSA in methanol effected the selective removal of one benzylidene group to give the corresponding diol in 70% yield based on recovered starting material. Selective protection of the primary hydroxyl group as its TBDMS ether followed by sequential protection of the secondary hydroxyl group as its MOM ether and removal of the TBDMS group with tetrabutylammonium fluoride (TBAF) gave 21 in 73% yield over three steps. Treatment of this alcohol with Dess-Martin periodinane provided the aldehyde which was reacted with methyltriphenylphosphonium bromide to yield the olefinic product 17 in 61% yield over two steps (Scheme 4).

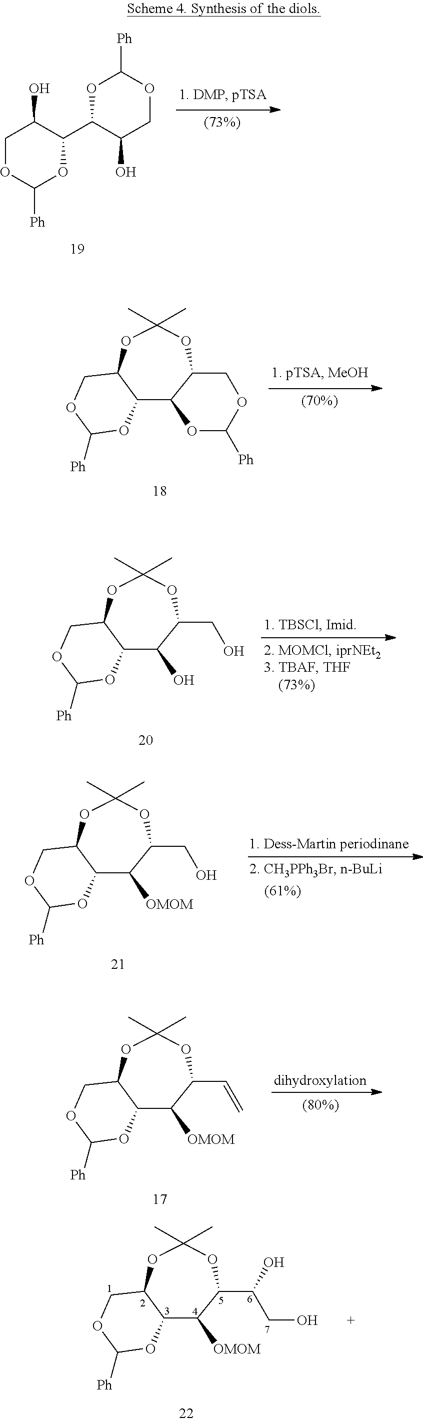

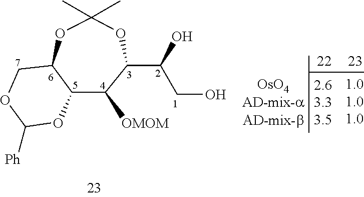

With compound 17 in hand, the inventors' next goal was to introduce the two hydroxyl groups. OsO₄-catalyzed dihydroxylation of 17 afforded compound 22 (Scheme 4) as the major product with a diastereomeric ratio of 22:23 of 2.6:1. Kishi's rule predicts that the relative stereochemistry between the pre-existing hydroxyl group and the adjacent newly-introduced hydroxyl group in the major product should be erythro.¹⁴ This result is also analogous to that obtained for dihydroxylation of a corresponding methylene acetal.¹²

Interestingly, AD-mix-α and AD-mix-β also afforded compound 22 as a major product, with a diastereomeric ratio of 3.3:1 and 3.5:1 (determined by 600 MHz ¹H NMR), respectively. The unsatisfactory selectivity can be explained by the steric hindrance imposed by the bicyclic structure, observed previously with a similar structure.¹⁵ The two isomers were separated by column chromatography and each was converted into its cyclic sulfate 16 or 26 as follows. The hydroxyl groups in 22 were protected as MOM ethers and the product was subjected to hydrogenolysis to effect removal of the benzylidene group and to yield the corresponding diol 24 in 72% yield over 2 steps. The cyclic sulfate 16 was then obtained by treatment of 24 with thionyl chloride in the presence of triethylamine to give the mixture of diastereomeric sulfites, followed by their oxidation with sodium periodate and ruthenium (III) chloride as a catalyst. A similar sequence of reactions with the diol 23 yielded the cyclic sulfate 26 (Scheme 5).

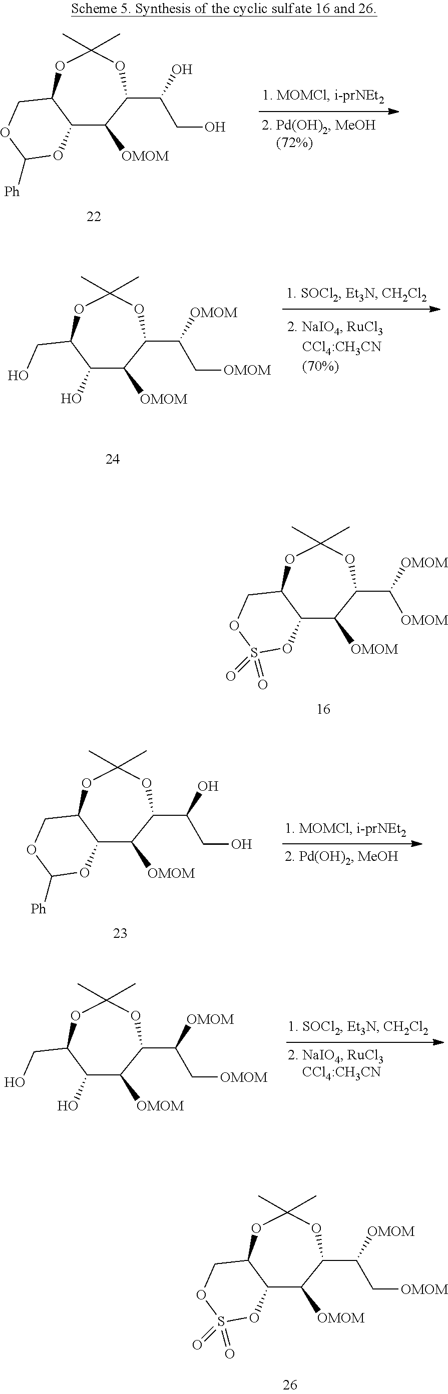

The target compounds were prepared by opening of the cyclic sulfates 16 and 26 by nucleophilic attack of the sulfur atom in 2,3,5-tri-O-p-methoxybenzyl-1,4-anhydro-4-thio-D-arabinitol 7.¹¹ Reactions were carried out at 72° C. in 1,1,1,3,3,3-hexafluoro-2-propanol (HFIP) containing K₂CO₃¹⁶ for 6 days to give the sulfonium salts 27 and 28 in 65 and 57% yield, respectively. Finally, deprotection of the coupled products 28 and 27 using aqueous 30% trifluoroacetic acid (TFA) at 50° C. gave the desired compounds 4 and 6 in 91 and 93% yields, respectively (Scheme 6).

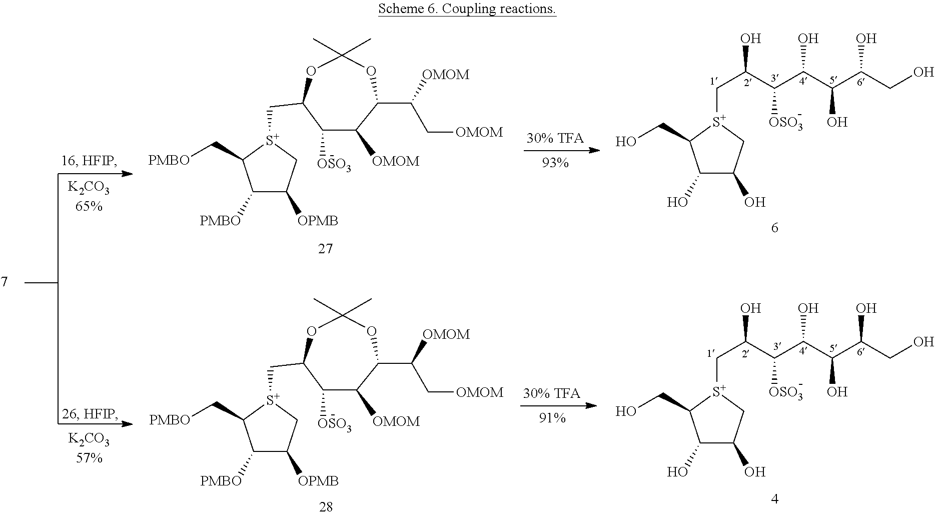

Comparison of the ¹H and ¹³C NMR spectra of kotalanol 4 with those reported¹² revealed identical data and served, therefore, to confirm the stereochemistry at C-6′, and, by inference, the stereochemistry at C-2 in each of 22 and 23.

The inventors measured the inhibitory activities of compounds 4 and 6 against the N-terminus of recombinant human maltase glucoamylase (ntMGA), a critical intestinal glucosidase for processing starch-derived oligosaccharides into glucose. The stereoisomer 6 of kotalanol 4 inhibited ntMGA with a Ki value of 0.20±0.02 μM; this compares to a Ki value for kotalanol of 0.19±0.03 μM,¹⁷ and Ki values of 0.10±0.02 μM and 0.13±0.02 μM for other stereoisomers of 4 with opposite configurations at C-5′ or both C-5′ and C-6′, respectively.¹⁵ The configurations at C-5′ and C-6′ are not critical for dictating enzyme inhibitory activity against ntMGA.

### 2.0 Nitrogen and Selenium Analogues of Kotalanol 4 and De-O-Sulfonated Kotalanol 5

The inventors have previously synthesized several analogues of salacinol 2 and studied their structure activity relationship (SAR) with human intestinal maltase glucoamylase (MGA).₁₁ Some of the modifications have included replacement of the ring sulfur heteroatom by the cognate atoms nitrogen^{18, 19} and selenium,²⁰ change of the configurations of the stereogenic centers, and extension of the acyclic side chain.²¹ Some of these compounds have shown higher or comparable inhibitory activities against MGA in vitro compared to acarbose and miglitol, two anti-diabetic drugs that are currently in use for the treatment of type-2 diabetes.^{17, 22} The acyclic side chain-extension studies of salacinol 2 led the inventors to predict the possible stereochemical pattern of the acyclic side chain in kotalanol 4, for which the absolute stereostructure was not determined at the time of its isolation. Recently, the inventors have proved the absolute stereostructure of kotalanol 4 and de-O-sulfonated kotalanol (5) by total syntheses.¹² In the case of salacinol 2, the substitution of the ring sulfur atom by nitrogen (ghavamiol, 30, IC₅₀=high mM range,²³ Chart 3) resulted in a dramatic decrease in inhibitory activity against MGA (compare the Kᵢ value of salacinol, 0.19 μM²²), whereas substitution by selenium (blintol, 31, Kᵢ=0.49 μM,²² Chart 3) did not affect its inhibitory activity appreciably.

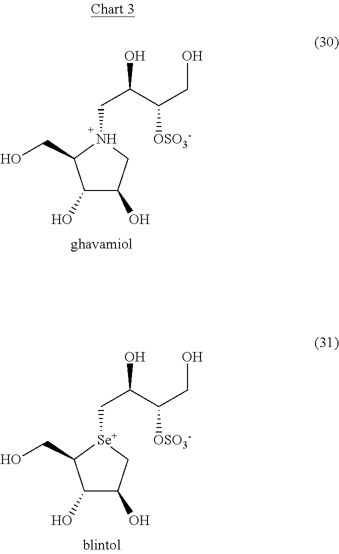

It is of interest, therefore, to study the effect of heteroatom substitution on the inhibitory activities of kotalanol 4 and de-O-sulfonated kotalanol 5, both having a 3-carbon extended acyclic side chain compared to salacinol 2. The syntheses of the nitrogen (32 and 33) and selenium (34 and 35) congeners of kotalanol and de-O-sulfonated kotalanol (Chart 4) and their evaluation as glucosidase inhibitors against MGA were performed. Since, de-O-sulfonated kotalanol 5 was found to be more active than kotalanol 4 itself,^{10, 24} the inventors have also converted two biologically active diastereomers 36 and 37 of kotalanol¹⁵ into their corresponding de-O-sulfonated analogues 38 and 39, respectively (Chart 5), and studied their inhibitory properties against MGA.

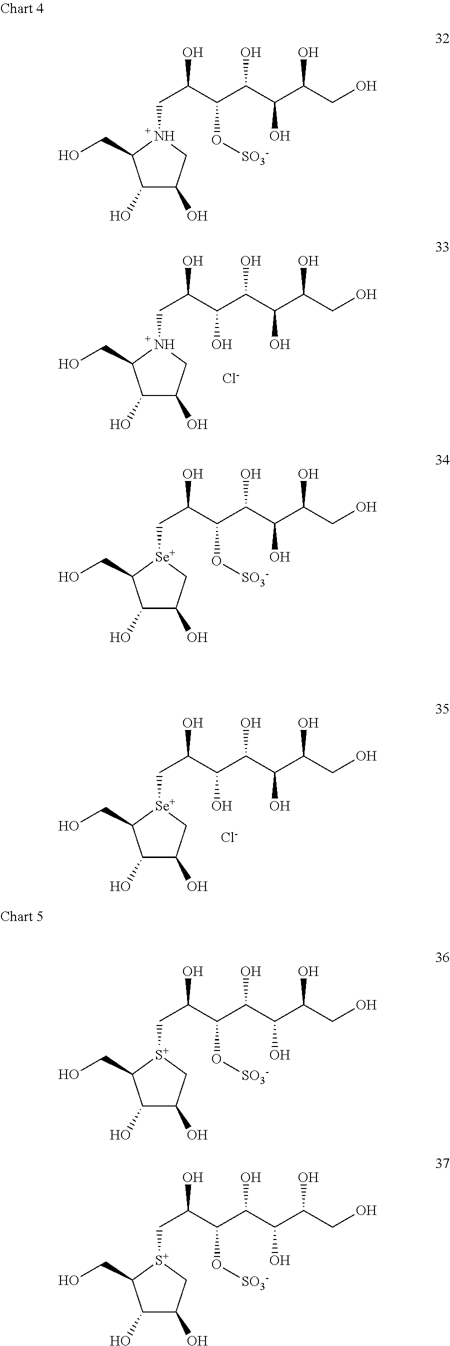

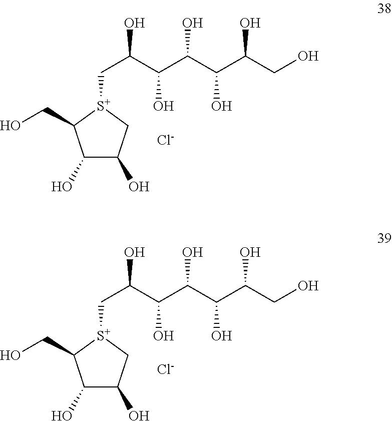 [SMILES: OC[C@@H]1[C@@H](O)[C@H](O)C[S@@+]1C[C@@H](O)[C@@H](O)[C@H](O)[C@@H](O)[C@@H](O)CO ; [Cl-] ; OC[C@@H](O)[C@H](O)[C@@H](O)[C@H](O)[C@H](O)C[S@+]1C[C@@H](O)[C@H](O)[C@H]1CO ; [Cl-]]

The required para-methoxybenzyl (PMB)-protected D-iminoarabinitol (40)²⁵ and D-selenoarabinitol (41)²⁶ were prepared by methods described in the inventors' earlier work. The required cyclic sulfate (42) was obtained from D-perseitol as reported earlier.²⁷

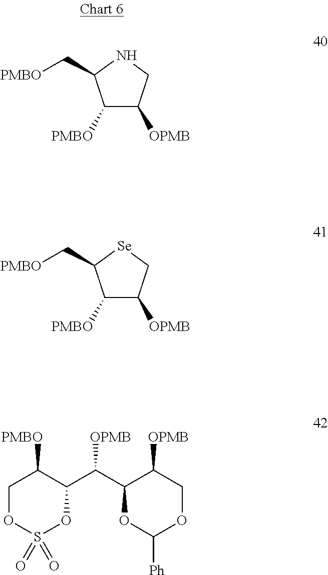

The synthesis of the nitrogen analogue 32 of kotalanol was examined first. The coupling reaction of the iminoarabinitol 40 with the cyclic sulfate 42 proceeded smoothly under our optimized reaction conditions (sealed tube, acetone, K₂CO₃, 60° C.).²⁵ The coupled product 43 was purified by short column chromatography, but was deemed to be unstable, probably due to the partial removal of PMB protecting groups, as confirmed by the formation of a more polar spot on TLC. Hence, without any further characterization, the coupled product 43 was taken on to the next step, namely removal of the PMB and benzylidene protecting groups using TFA/CH₂Cl₂, as shown in Scheme 7.

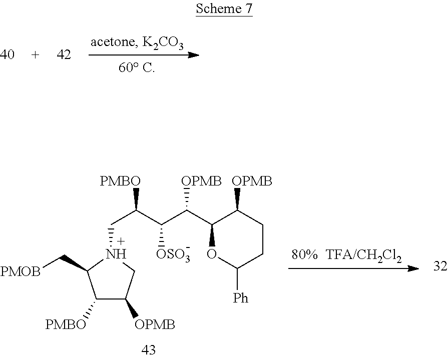

Similarly, the selenium analogue 34 of kotalanol was obtained from selenoarabinitol 41 and the cyclic sulfate 42 using the inventors' optimized reaction conditions (sealed tube, HFIP, K₂CO₃, 70° C.).²⁵ As observed in previous work from the inventors' laboratory,²⁰ during the coupling reaction of D-selenoarabinitol 41 with the cyclic sulfate 42, along with the desired coupled product (44, 40% yield), a considerable amount of the undesired diastereomer (45, 26% yield), with respect to the selenium center, was also formed. The undesired diastereomer 45 was conveniently separated from the desired coupled product 44 by column chromatography. Once again, the removal of the PMB and benzylidene protecting groups was achieved in one pot using TFA/CH₂Cl₂. Thus, compounds 44 and 45 upon deprotection gave 34 and 46, respectively, as final products.

The absolute configuration at the stereogenic selenium center in compound 34 was established by means of a 1D-NOESY experiment. A correlation between H-4 and H-1′ a confirmed that they are syn-facial. In the case of compound 46, correlation of H-1b with H-3 and also with H-1′ a confirmed that they all are syn facial, thus establishing the absolute configuration at the selenium center as S (Scheme 8). Compound 46 differs from 34 only with respect to the configuration at the stereogenic selenium center. Hence, this compound 46 served as a probe of the importance of the R configuration at the positively charged ring heteroatom for inhibitory activity; all of the naturally-occurring compounds 1-5 have the R configuration at the stereogenic sulfur center. In the case of the nitrogen analogue 32, the absolute configuration at the ammonium center was assigned as R by analogy with the inventors' previous work,^{18, 25} since a NOESY experiment was not possible owing to the broad, overlapping signals at neutral pH.

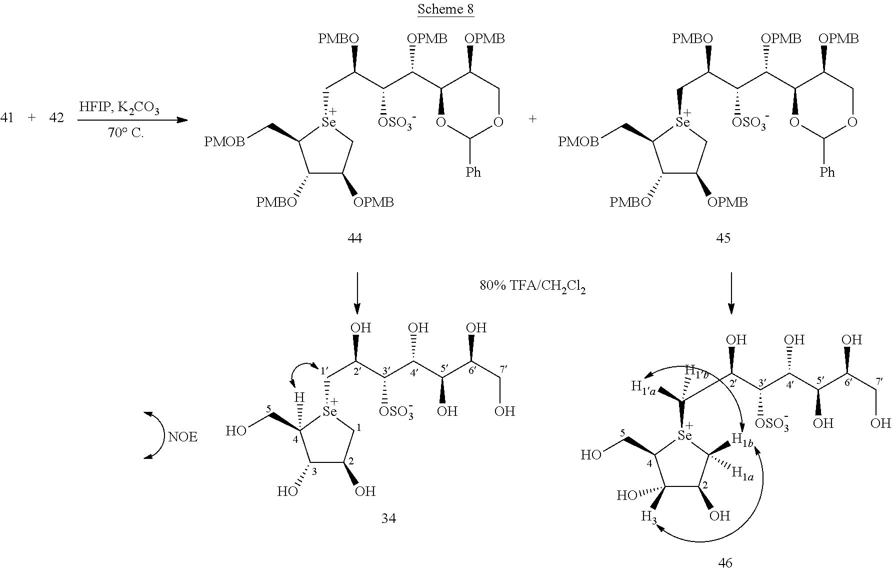

With the sulfated compounds in hand, the inventors turned next to the synthesis of the corresponding de-O-sulfonated analogues. Compounds 32, 34, 36,¹⁵ and 37¹⁵ were converted into their corresponding de-O-sulfonated compounds 33, 35, 38, and 39 respectively, in a two step process, first treatment with 5% methanolic HCl,⁹ followed by treatment with Amberlyst-A26 (chloride resin) in MeOH, as shown in the general Scheme 9. Similarly, compound 46 was also converted into the corresponding de-O-sulfonated compound 47 (Chart 6).

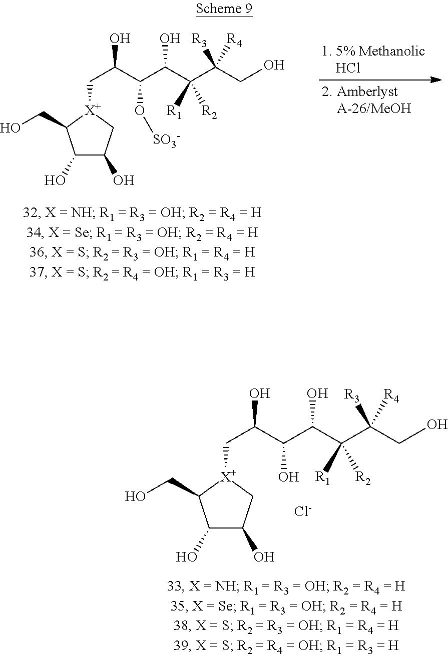

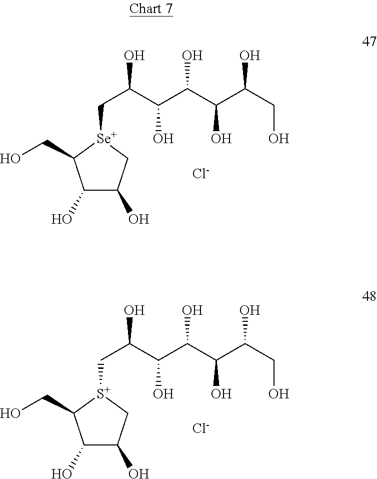 [SMILES: OC[C@@H]1[C@@H](O)[C@H](O)C[Se@+]1C[C@@H](O)[C@@H](O)[C@H](O)[C@H](O)[C@@H](O)CO ; [Cl-] ; OC[C@@H](O)[C@@H](O)[C@@H](O)[C@H](O)[C@H](O)C[S@+]1C[C@@H](O)[C@H](O)[C@H]1CO ; [Cl-]]

The inhibitory activities of the synthesized compounds (32-35, 38, 39, 46 and 47) against MGA was determined as summarized in Table 1 below. In addition, the inventors also determined the enzyme inhibitory activity of compound 48, a diastereomer of de-O-sulfonated kotalanol, that was previously synthesized (Chart 7).¹² Except for the nitrogen analogue of kotalanol 32, all of the compounds synthesized in this study show greater inhibitory activities than acarbose, an antidiabetic agent that is currently approved for the treatment of type-2 diabetes (Table 1).²² In general, de-O-sulfonation leads to an increase in inhibitory activity compared to the parent sulfated compounds. Interestingly, in the case of the nitrogen analogue of kotalanol 32, de-O-sulfonation resulted in a very large increase in inhibitory activity (compare Kᵢ values of compounds 32 and 33, Table 1). These results also indicate that the substitution of the ring sulfur atom by selenium does not confer any significant advantage (kotalanol, X=Se: Kᵢ=80 nM. X═S: Kᵢ=190 nM) and de-O-sulfonated kotalanol (X=Se: Kᵢ=20 nM. X═S: Kᵢ=30 nM)). Interestingly, substitution of the ring sulfur atom by nitrogen in compound 32 is detrimental to inhibitory activity (Kᵢ=90 μM), whereas it does not have any significant change on the inhibitory activity of the nitrogen analogue of de-O-sulfonated kotalanol 33 (Kᵢ=61 nM). The significant decrease in the inhibitory activity of the nitrogen analogue 32 of kotalanol relative to kotalanol 4 deserves comment. Interestingly, this trend was also observed with ghavamiol 30, the nitrogen analogue of salacinol, relative to salacinol 2. Without being bound by any particular theory, the inventors hypothesize, based on recent crystallographic work with salacinol and kotalanol derivatives,¹⁷ that the positioning of the sulfate anion of 32 in a hydrophobic pocket in the active site is more sterically compromised than in the sulfur congener 4. Relief of this steric interaction by de-O-sulfonation to give 33 apparently relieves this interaction, and gives a compound that is just as active as its sulfur congener 5. The inventors note also that the R configuration at the stereogenic heteroatom center, as exhibited by all of the natural compounds (1-5) isolated so far, is essential for inhibitory activity; thus, the inhibitory activities of compounds 46 and 47, bearing the S configuration at the stereogenic selenium center, are considerably less than those of their corresponding diastereomers with the R configuration, 34 and 35, respectively. As predicted, the de-O-sulfonated compounds, 38 and 39, are found to be more active compared to the parent compounds, 36 and 37, respectively.

TABLE 1
Experimentally determined Kᵢ values^{a}

| Inhibitor | Kᵢ (nM) |
|---|---|
| 4 | 190 ± 30⁽¹⁷⁾ |
| 5 | 30 ± 10⁽¹⁷⁾ |
| 32 | 90000 ± 6000 |
| 33 | 61 ± 5 |
| 34 | 80 ± 6 |
| 35 | 20 ± 3 |
| 36 | 130 ± 20⁽¹⁵⁾ |
| 37 | 100 ± 20⁽¹⁵⁾ |
| 38 | 24 ± 2 |
| 39 | 26 ± 2 |
| 46 | 7200 ± 700 |
| 47 | 830 ± 70 |
| 48 | 17 ± 1 |
| acarbose | 62000 ± 13000⁽²²⁾ |

^{a}Analysis of MGA inhibition was performed using maltose as the substrate

### 3.0 De-O-Sulfonated Ponkoranol and its Stereoisomer

As indicated above, several de-O-sulfonated kotalanol derivatives have been found to be more biologically active in in vitro tests than their parent compounds. The same finding has been demonstrated by other salacinol homologues.

Minami et al.²⁸ recently reported the isolation of a thiosugar sulfonium-alkoxide inner salt (49), neosalacinol, from Salacia reticulata.

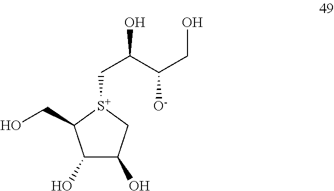 [SMILES: [O-][C@@H](CO)[C@H](O)C[S@+]1C[C@@H](O)[C@H](O)[C@H]1CO]

However, Yoshikawa et al.²⁹ have shown that this compound is de-O-sulfonated salacinol (50); its synthesis employed the coupling reaction of thioarabinitol 51³⁰ with a protected epoxide 52 (Scheme 10).

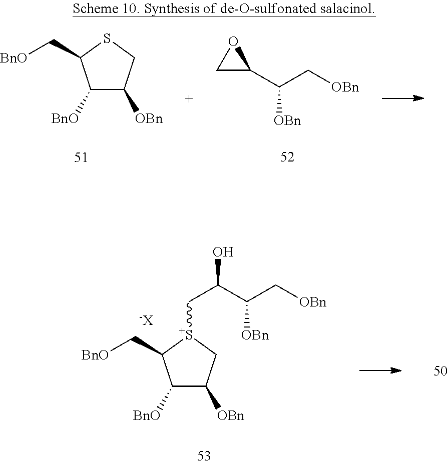

As indicated above, comparison of the inhibitory activities of de-O-sulfonated salacinol 50 vs. salacinol 2 and de-O-sulfonated kotalanol 5 vs. kotalanol 4 against rat intestinal α-glucosidases (maltase, sucrase and isomaltase) revealed that the desulfonated analogues were either equivalent or better inhibitors than the parent compounds.^{9, 24, 31}

In view of these findings, the inventors further investigated whether de-O-sulfonated ponkoranol 54 or its stereoisomer 55 (Chart 8) would be more potent inhibitors than ponkoranol itself. Other studies described above with regard to kotalanol analogues had suggested that the configuration at C-5′ was not critical for inhibitory activity.^{15, 17}

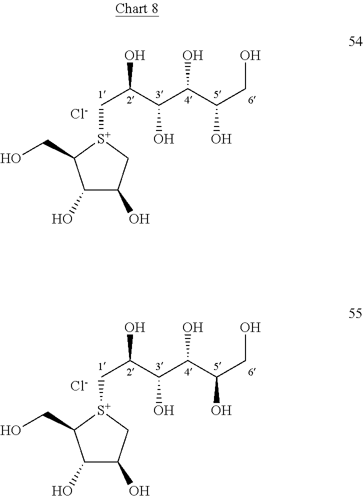 [SMILES: OC[C@@H]1[C@@H](O)[C@H](O)C[S@@+]1C[C@@H](O)[C@@H](O)[C@H](O)[C@@H](O)CO ; [Cl-] ; OC[C@@H](O)[C@@H](O)[C@H](O)[C@H](O)C[S@+]1C[C@@H](O)[C@H](O)[C@H]1CO ; [Cl-]]

The sulfonium ions A could be synthesized by alkylation of an appropriately protected 1,4-anhydro-4-thio-D-arabinitol B at the ring sulfur atom with agent C. The desired stereochemistry at C-5′ could be obtained by choice of either glucose or mannose as starting material (Scheme 11).

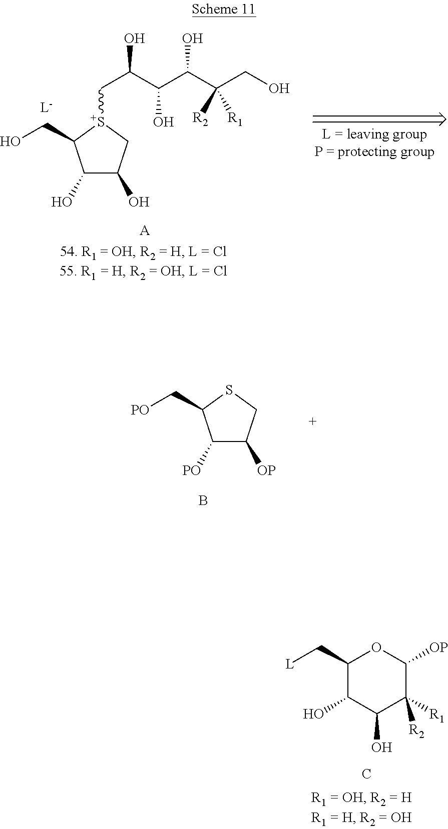

Initially, the S-alkylation of thioarabinitol 51 with methyl 6-iodo-β-D-glucopyranoside 56 ³² in CH₃CN using AgBF₄ at 65° C. was examined, based on the procedure that has been reported for S-alkylation with simple alkyl chains (Scheme 12). ³³ No product formation and decomposition of the starting material was observed by TLC; the reaction in 1,1,1,3,3,3-hexafluoroisopropanol (HFIP) as a solvent was also unsuccessful.

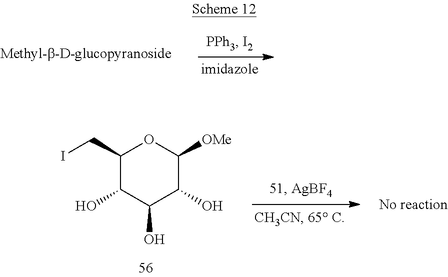 [SMILES: CO[C@@H]1O[C@H](CI)[C@@H](O)[C@H](O)[C@H]1O]

The inventors chose to replace the iodo group of compound 56 with a p-toluenesulfonyl ester (compound 57). The coupling reaction in HFIP at 70° C. now proceeded smoothly and yielded the sulfonium ion 58 (Scheme 13). However, attempts to hydrolyze the methyl glycoside were not successful and decomposition of the product was observed.

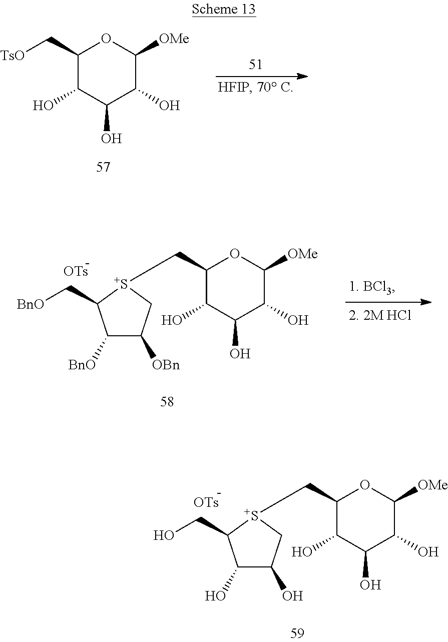 [SMILES: CO[C@@H]1O[C@H](COS(=O)(=O)c2ccc(C)cc2)[C@@H](O)[C@H](O)[C@H]1O ; CO[C@@H]1O[C@H](CC[S+]2C[C@@H](OCc3ccccc3)[C@H](OCc3ccccc3)[C@H]2COCc2ccccc2)[C@@H](O)[C@H](O)[C@H]1O ; CO[C@@H]1O[C@H](CC[S+]2C[C@@H](O)[C@H](O)[C@H]2CO)[C@@H](O)[C@H](O)[C@H]1O]

Therefore, a benzyl glycoside was chosen as a protecting group at the anomeric position to ensure its facile removal after the coupling reaction. Thus, benzyl 6-O-p-toluene-sulfonyl-β-D-gluco or manno-pyranoside 60 and 61 were readily prepared from D-glucose and D-mannose, respectively according to literature procedures. ³⁴⁻³⁶ The thioether 51 was reacted with 60 in HFIP containing K₂CO₃ ¹⁶ to give the protected sulfonium ion 62 in 52% yield (Scheme 14). The benzyl groups were then removed by treatment with boron trichloride at −78° C. in CH₂Cl₂. During the course of deprotection, the p-toluenesulfonate counterion was partially exchanged with chloride ion. Similar results were observed in previous work from the inventors' laboratory. ³³ Hence, after removal of the benzyl groups, the product was subsequently treated with Amberlyst A-26 resin (chloride form) to completely exchange the p-toluenesulfonate counterion with chloride ion. Finally, the crude product was reduced with NaBH₄ to provide the desired de-O-sulfonated ponkoranol 54 in 48% yield over 3 steps (Scheme 14).

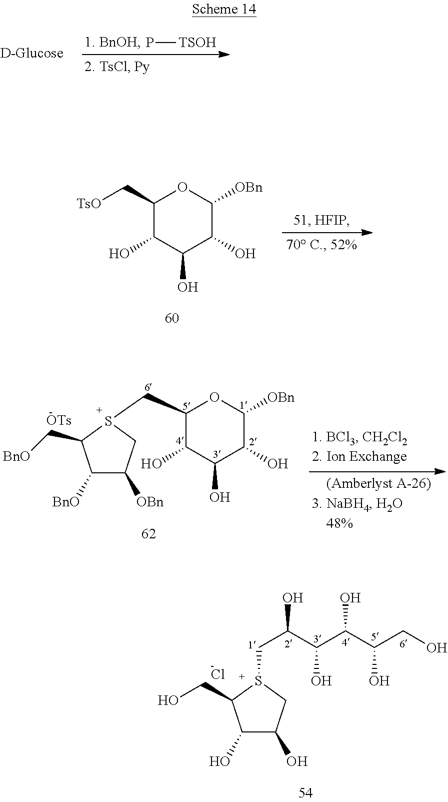

The other diastereomer was obtained similarly. Thus, compound 61 was reacted with the thioether 51 to give the protected sulfonium ion 63 in 47% yield which was converted, as before, to the desired compound 55 in 41% yield over 3 steps (Scheme 15).

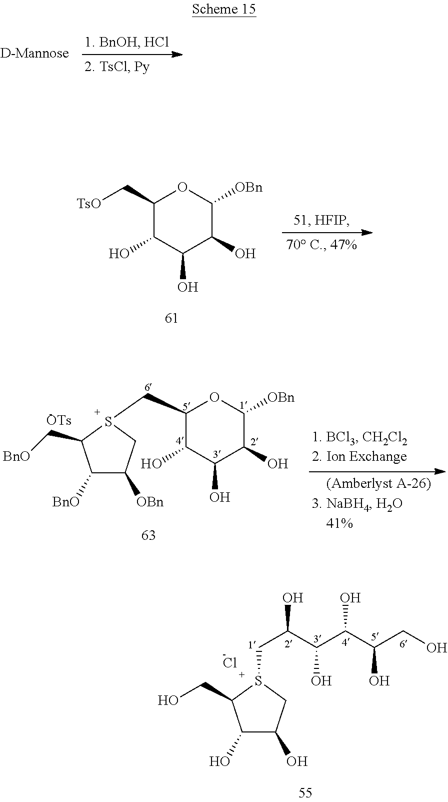

Finally, the inventors determined the inhibitory activities of compounds 54 and 55 against the N-terminus of recombinant human maltase glucoamylase (ntMGA), a critical intestinal glucosidase for processing starch-derived oligosaccharides into glucose. The de-O-sulfonated ponkoranol 54 and its stereoisomer 55 inhibited ntMGA with Ki values of 43±3 and 15±1 nM, respectively. This compares to a Ki value for de-O-sulfonated kotalanol of 30±1 nM.²³ The configuration at C-5′ is thus not critical for dictating enzyme inhibitory activity against ntMGA and, furthermore, extension of the acyclic carbon chain beyond six carbons is not beneficial.

### 4.0 Selenium Analogue of The C-5′ Epimer of De-O-Sulfonated Ponkoranol

The selenoether 64²⁰ was reacted with 61 in HFIP containing K₂CO₃ to give the protected selenonium ion 65 in 45% yield (Scheme 16). The benzyl groups were then removed by treatment with boron trichloride at −78° C. in CH₂Cl₂. During the course of deprotection, the p-toluenesulfonate counterion was partially exchanged with chloride ion. Hence, after removal of the benzyl groups, the product was subsequently treated with Amberlyst A-26 resin (chloride form) to completely exchange the p-toluenesulfonate counterion with chloride ion. Finally, the crude product was reduced with NaBH₄ to provide the desired C-5′ epimer of the selenium analogue of de-O-sulfonated ponkoranol 66 (Scheme 16).

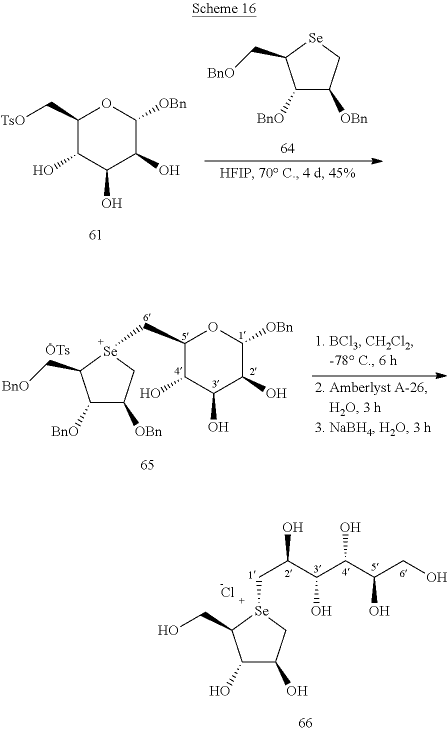

Compounds that are inhibitors of glycosidases such as MGA may be used in the treatment of diabetes. Compounds that are selective inhibitors of intestinal glucosidases (i.e. which do not inhibit amylase activity) may be as clinically effective in treating diabetes as agents such as acarbose which inhibit pancreatic α-amylase preferentially; however, because such compounds interfere less with digestion of starch by pancreatic α-amylase, they may have less side effects.³⁷ For example, one study has found that at equivalent dosages, the incidence of flatulence as an adverse event in response to administration of glucosidase inhibitors may be reduced with miglitol, which does not inhibit amylase activity, as compared with acarbose.^{37, 38}

A method for treating diabetes in an affected patient may include the step of administering a therapeutically effective amount of a compound that is a glucosidase inhibitor. The glucosidase inhibitor may be one or more of compounds 6, 32, 33, 34, 35, 36, 37, 38, 39, 54, 55 or 66 described herein.

### EXAMPLES

The following examples will further illustrate the invention in greater detail although it will be appreciated that the invention is not limited to the specific examples.

### Example 1.0

### Synthesis of Kotalanol 4 and its Stereoisomer 6

General:

Optical rotations were measured at 23° C. ¹H and ¹³C NMR spectra were recorded at 600 and 150 MHz, respectively. All assignments were confirmed with the aid of two-dimensional ¹H, ¹H (COSYDFTP) or ¹H, ¹³C (INVBTP) experiments using standard pulse programs. Column chromatography was performed with Silica 60 (230-400 mesh). High resolution mass spectra were obtained by the electrospray ionization method, using an Agilent 6210 TOF LC/MS high resolution magnetic sector mass spectrometer.

Enzyme Inhibition Assays:

Compounds 4 and 6 were tested for inhibition of ntMGA, as previously described.¹⁵

### 1,3,4,6-di-O-Benzylidene-2,5-O-isopropylidene-D-mannitol (18)

Compound 19 (9.30 g, 26.00 mmol) was dissolved in 2,2-di-methoxypropane (150 mL), PTSA (1.50 g, 0.3 eq) was added, and the mixture was stirred at room temperature under reduced pressure for 1 hour. The reaction mixture was quenched by addition of Et₃N to pH>9. The reaction mixture was concentrated under vacuum to give a white solid which was dissolved in CHCl₃ (200 mL) and washed with water (3×50 mL). The separated organic layer was dried over Na₂SO₄, concentrated, and the residue was purified by column chromatography with EtOAc/Hexanes (1:4) as eluent to afford 18 as a white solid (7.55 g, 73%). Mp 160-162° C.; [α]_{D}²³=−83°, (c=1.1, CH₂Cl₂). ¹H NMR (CDCl₃) δ 7.54-7.37 (10H, m, Ar), 5.54 (2H, s, 2CH-Ph), 4.24 (2H, dd, J_{1a,1b}=10.8, J_{2,1}=5.3 Hz, H-1), 3.95-3.91 (2H, m, H-6a, H-5), 3.84-3.80 (2H, m, H-3, H-4), 3.74 (2H, t, H-2, H-6b), 1.42 (6H, s, 2Me). ¹³C NMR (CDCl₃) δ 137.5 (CMe₂), 129.9-126.2 (m, Ar), 100.7 (CH-Ph), 82.2 (C-3, C-4), 69.4 (C-1, C-6), 61.7 (C-2, C-5), 24.4 (2Me). HRMS Calcd for C₂₃H₂₇O₆ (M+H): 399.1802. Found: 399.1809.

### 1,3-O-Benzylidene-2,5-O-isopropylidene-D-mannitol (20)

To a solution of compound 18 (7.50 g, 18.84 mmol) in MeOH (300 mL), was added PTSA (300 mg), and the reaction was stirred at room temperature for 30 min. The reaction mixture was then quenched by addition of Et₃N to pH>9, and the solvent was removed under vacuum to give a solid. The solid was dissolved in CH₂Cl₂ (100 mL) and washed with water (50 mL). The organic solution was dried (Na₂SO₄), concentrated, and the crude product was purified through a silica column with EtOAc/Hexanes (1:1) as eluent to yield 20 as a foam (4.1 g, 70%). [α]_{D}²³=−15°, (c=1, CH₂Cl₂).¹H NMR (CDCl₃) δ 7.42-7.30 (5H, m, Ar), 5.40 (1H, s, CH-Ph), 4.12 (1H, dd, J_{1a,1b}=10.8, J_{1a,2}=5.5 Hz, H-1a), 3.81 (1H, dd, J_{6a,6b}=10.9, J_{6a,5}=4.3 Hz, H-6a), 3.76-3.72 (2H, m, H-3, H-5), 3.66 (1H, m, H-6b), 3.60-3.53 (2H, m, H-4, H-1b), 3.43 (1H, t, J_{1,2}=8.9 Hz, H-2), 2.23 (2H, b, 20H), 1.30 (6H, s, 2Me).¹³C NMR (CDCl₃) δ 137.3 (CMe₂), 129.3-101.7 (m, Ar), 101.1 (CH-Ph), 85.2 (C-2), 73.9 (C-4), 70.3 (C-5), 69.3 (C-1), 63.6 (C-6), 61.2 (C-3), 24.8, 24.6 (2Me). HRMS Calcd for C₁₆H₂₃O₆ (M+H): 311.1489. Found: 311.1487.

### 1,3-O-Benzylidene-2,5-O-isopropylidene-4-O-methoxymethyl-D-mannitol (21)

To a solution of 20 (6.80 g, 21.93 mmol) in DMF (125 mL) was added imidazole (4.47 g, 65.81 mmol). The reaction was cooled in an ice bath, TBDMSCl (3.79 g, 24.13 mmol) was added portionwise, and the mixture was stirred at 0° C. under N₂ for 2 hours. The reaction was quenched by the addition of ice-water, and the reaction mixture was extracted with Et₂O (3×75 mL). The combined organic solvents were dried (Na₂SO₄) and concentrated to give the crude product which was used directly in the next step without further purification. The crude product was dissolved in DMF (60 mL), and i-Pr₂NEt (26 mL, 150.75 mmol) and MOMCl (5.7 mL, 75.38 mmol) were added. The reaction mixture was heated at 60° C. overnight, then quenched with ice, and extracted with ether (3×50 mL). The organic solution was dried (Na₂SO₄) and concentrated to give a crude product. The crude residue was dissolved in THF (100 mL), TBAF (1.0 M solution in THF, 13.8 mL, 24.12 mmol) was added, and the reaction mixture was stirred at room temperature. After 4 hours it was concentrated and the residue was purified by flash chromatography (EtOAc/Hexanes (1:3)) to yield 21 as a white solid (5.67 g, 73%). Mp 65-67° C.; [α]_{D}²³=+22 (c=1, MeOH). ¹H NMR (CDCl₃) δ 7.49-6.37 (5H, m, Ar), 5.50 (1H, s, CH-Ph), 4.93, 4.73 (2H, 2d, J_{A,B}=6.4 Hz, CH₂OMe), 4.20 (1H, dd, J_{1a,1b}=10.9, J_{1a,2}=5.5 Hz, H-1a), 3.89-3.79 (4H, m, H-2, H-5, H-6a,b), 3.74 (1H, t, J_{3,4}=J_{5,4}=8.1 Hz, H-4), 3.69-3.65 (2H, m, H-1b, H-3), 3.40 (3H, s, OMe), 2.69 (1H, t, J_{6,OH}=8.5 Hz, OH), 1.41, 1.38 (6H, 2s, 2Me).¹³C NMR (CDCl₃) δ 137.5 (CMe₂), 128.9-101.5 (m, Ar), 100.9 (CH-Ph), 98.6 (CH₂—OMe) 85.3 (C-3), 78.2 (C-4), 70.4 (C-5), 69.5 (C-1), 63.1 (C-6), 61.3 (C-2), 56.4 (OMe), 24.7, 24.4 (2Me). HRMS Calcd for C₁₈H₂₇O₇ (M+H): 355.1751. Found: 355.1741.

### 1,3-O-Benzylidene-2,5-O-isopropylidene-4-O-methoxymethyl-D-manno-hep-6-enitol (17)

Compound 21 (2.60 g, 7.34 mmol) was dissolved in CH₂Cl₂ (50 mL) and NaHCO₃ (2.77 g, 33.03 mmol) and Dess Martin periodinane (3.73 g, 8.81 mmol) were added. The reaction mixture was stirred for 2 hours at room temperature, diluted with ether (100 mL), and poured into saturated aqueous NaHCO₃ (100 mL) containing a seven fold excess of Na₂S₂O₃. The mixture was stirred to dissolve the solid, and the ether layer was separated and dried over Na₂SO₄. The ether was removed to give the aldehyde that was further dried under high vacuum for 1 hour. Methyltriphosphonium bromide (2.99 g, 8.80 mmol) in dry THF (15 mL), was cooled to −78° C. and n-BuLi (n-hexane solution, 14.67 mmol) was added dropwise under N₂. The reaction mixture was stirred at the same temperature for 1 hour, and a solution of the previously made aldehyde in THF (10 mL) was added. The resulting mixture was allowed to warm to room temperature and was stirred overnight. The reaction was quenched by the addition of acetone (1.5 mL), and the mixture was extracted with ether (3×100 mL). The combined organic layers were washed with brine, dried (Na₂SO₄), and concentrated in vacuo. Chromatographic purification of the crude product (EtOAc/Hexanes (1:10)) gave 17 as a foam (1.56 g, 61%). [α]_{D}²³=+4 (c=0.5, CH₂Cl₂). ¹H NMR (CDCl₃) δ 7.50-7.36 (5H, m, Ar), 6.05 (1H, ddd, J_{5,6}=6.1, J_{6,7b}=10.5, J_{6,7a}=16.6 Hz, H-6), 5.51 (1H, s, CH-Ph), 5.39 (1H, ddd, J_{7b,7a}=17.1, J_{6,7a}=3.3, J_{5,7a}=1.5 Hz, H-7a), 5.36 (1H, ddd, J_{7a,7b}=10.7, J_{6,7b}=3.1, J_{5,7b}=1.5 Hz, H-7b), 5.27, 5.26 (2H, 2d, J_{A,B}=6.25 Hz, CH₂OMe), 4.25 (1H, m, H-5), 4.20 (1H, dd, J_{1a,1b}=10.8, J_{1a,2}=5.4 Hz, H-1a), 3.90 (1H, dt, J_{2,3}=5.4, J_{2,1}=9.9 Hz, H-2), 3.68 (2H, m, H-3, H-1b), 3.56 (1H, dd, J_{3,4}=8.1, J_{4,5}=9.7 Hz, H-4), 3.33 (3H, s, OMe), 1.40, 1.37 (6H, 2s, 2Me). ¹³C NMR (CDCl₃) δ 137.6 (CMe₂), 136.2 (C-6), 128.9-101.3 (m, Ar), 116.8 (C-7), 100.7 (CH-Ph), 97.9 (CH₂OMe), 85.5 (C-3), 80.2 (C-4), 71.1 (C-5), 69.6 (C-1), 61.4 (C-2), 56.4 (OMe), 24.8, 24.1 (2Me). HRMS Calcd for C₁₉H₂₆NaO₆ (M+Na): 373.1622. Found: 373.1606.

### 1,3-O-Benzylidene-2,5-O-isopropylidene-4-O-methoxymethyl-D-glycero-D-manno-heptitol (22)

To a solution of 17 (2.00 g, 5.71 mmol) in acetone:water (9:1, 6 mL) at room temperature were added NMO (N-methylmorpholine-N-oxide) (735 mg, 6.28 mmol) and OsO₄ (40 mg, 2.5 wt % solution in 2-methyl-2-propanol). The reaction mixture was stirred at room temperature for 48 hours before it was quenched with a saturated solution of NaHSO₃. After being stirred for an additional 15 minutes the reaction mixture was extracted with ethyl acetate and the organic layer was washed with water and brine, dried (Na₂SO₄), and concentrated in vacuo. The crude material was purified by column chromatography on silica gel (MeOH/CH₂Cl₂ (1:100)) to give 22 (1.27 g, 58%) and 23 (0.48 g, 22%) as foams. [α]_{D}²³=+5.8° (c=4.6, MeOH). ¹H NMR (MeOD) δ 7.49-7.36 (5H, m, Ar), 5.54 (1H, s, CH-Ph), 4.82 (1H, s, CH₂OMe), 4.13 (1H, dd, br, H-1a), 4.00 (1H, br, q, H-6), 3.87-3.77 (3H, m, H-4, H-5, H-2), 3.68-3.55 (4H, H-1b, H-3, H-7a, H-7b), 3.32 (3H, s, OMe), 1.39, 1.34 (6H, 2s, 2Me).¹³C NMR (MeOD) δ 138.0 (CMe₂), 128.4-101.1 (m, Ar), 100.8 (CH-Ph), 97.7 (CH₂OMe), 85.3 (C-4), 77.1 (C-2), 69.2 (C-6), 69.1 (C-5), 69.0 (C-1), 62.3 (C-7), 61.1 (C-3), 55.3 (OMe), 23.5, 23.4 (2Me). HRMS Calcd for C₁₉H₂₉O₈ (M+H): 385.1857. Found: 385.1875.

### 5,7-O-Benzylidene-3,6-O-isopropylidene-4-O-methoxymethyl-D-glycero-D-galacto-heptitol (23)

[α]_{D}²³=−20° (c=0.1, MeOH). ¹H NMR (MeOD) δ 7.48-7.34 (5H, m, Ar), 5.51 (1H, s, CH-Ph), 4.49, 4.47 (2H, 2d, J_{A,B}=6.2 Hz, CH₂OMe), 4.13 (1H, dd, J_{7a,7b}=10.7, J_{6,7b}=5.4 Hz, H-7a), 4.08 (1H, m, H-2), 3.95 (1H, dd, J_{3,4}=9.7, J_{5,4}=2.8 Hz, H-4), 3.85 (1H, dd, J_{1a,1b}=11.4, J_{2,1a}=3.6 Hz, H-1a), 3.78 (1H, dt, J_{6,7}=9.9, J_{5,6}=5.4 Hz, H-6), 3.67-3.60 (4H, m, H-5, H-7b, H-1b, H-3), 3.35 (3H, s, OMe), 1.37, 1.36 (6H, 2s, 2Me).¹³C NMR (MeOD) δ 137.9 (CMe₂), 128.5-101.3 (m, Ar), 100.6 (CH-Ph), 97.7 (CH₂OMe), 86.0 (C-5), 78.2 (C-3), 72.1 (C-4), 71.3 (C-2), 69.1 (C-7), 61.3 (C-1), 61.0 (C-6), 55.6 (OMe), 23.6, 23.4 (2Me). HRMS Calcd for C₁₉H₂₉O₈ (M+H): 385.1857. Found: 385.1865.

### 2,5-O-isopropylidene-4,6,7-tri-O-methoxymethyl-D-glycero-D-manno-heptitol (24)

Compound 22 (580 mg, 1.51 mmol), was dissolved in DMF (20 mL) and i-Pr₂NEt (4.21 mL, 24.16 mmol) and MOMCl (0.9 mL, 12.08 mmol) were added. The reaction mixture was heated at 60° C. for 2 hours, then quenched with ice, and extracted with ether (3×30 mL). The organic solution was dried (Na₂SO₄) and concentrated to give a crude product that was further dried under high vacuum for 1 hour. The crude product was dissolved in MeOH (50 mL) and the solution was stirred with Pd(OH)₂ 20 wt % on carbon (520 mg) under 100 Psi of H₂ for 1 hour. The catalyst was removed by filtration through a bed of Celite, then washed with methanol. The solvents were removed under reduced pressure and the residue was purified by flash column chromatography (EtOAc/Hexanes (1.5:1)) to give 24 as a colorless syrup (420 mg, 72%).[α]_{D}²³=+48.0° (c=0.1, MeOH). ¹H NMR (MeOD) δ 4.90-4.63 (6H, m, 3CH₂OMe), 4.20 (1H, dd, br, H-6), 3.95 (1H, d, br, J_{4,5}=8.6 Hz, H-5), 3.86-380 (2H, m, H-1a, H-7a), 3.68-3.58 (3H, m, H-2, H-7b, H-1b), 3.45, 3.42, 3.36 (9H, 3s, 3OMe), 3.34 (2H, m, H-4, H-3), 1.35 (6H, s, 2Me). ¹³C NMR (CDCl₃) δ 100.7 (CMe₂), 98.4, 96.3, 95.5 (3CH₂OMe), 83.9 (C-4), 75.0 (C-6), 74.9 (C-3), 71.2 (C-2), 70.5 (C-5), 66.2 (C-7), 62.5 (C-1), 55.3, 54.5, 54.1 (3OMe), 22.6, 22.4 (2Me). HRMS Calcd for C₁₆H₃₃O₁₀ (M+H): 385.2068. Found: 385.2083.

### 3,6-O-isopropylidene-1,2,4-tri-O-methoxymethyl-D-glycero-D-galacto-heptitol (25)

Compound 25 was obtained as a colorless syrup (285 mg, 75%) from 23 (380 mg, 1 mmol) using the same procedure that was used to obtain 24. [α]_{D}²³=−30° (c=0.4, MeOH).¹H NMR (MeOD) δ 4.84-4.61 (6H, m, 3CH₂OMe), 4.08 (1H, ddd, J_{3,2}=1.3, J_{2,1a}=5.6, J_{2,1b}=7.2 Hz, H-2), 3.86-3.84 (2H, m, H-7a, H-3), 3.74 (1H, dd, J_{1a,1b}=9.5, J_{1a,2}=5.6 Hz, H-1a), 3.69 (1H, ddd, J_{6,5}=2.9, J_{6,7b}=6.8, J_{6,7a}=9.8 Hz, H-6), 3.60-3.55 (21-1, m, H-1b, H-7b), 3.45 (1H, m, H-5), 3.44, 3.38, 3.35 (9H, 3s, 3OMe), 3.34 (1H, m, H-4), 1.36, 1.32 (6H, 2s, 2Me). ¹³C NMR (CDCl₃) δ 100.1 (CMe₂), 97.8, 96.8, 95.9 (3CH₂OMe), 83.3 (C-5), 75.1 (C-2), 73.8 (C-4), 70.3 (C-6), 67.8 (C-3), 65.9 (C-1), 61.8 (C-7), 54.3, 54.1, 53.7 (3OMe), 22.9, 22.8 (2Me). HRMS Calcd for C₁₆H₃₃O₁₀ (M+H): 385.2068. Found: 385.2067.

### 2,5-O-isopropylidene-4,6,7-tri-O-methoxymethyl-D-glycero-D-manno-heptitol-1,3-cyclic sulfate (16)

A mixture of 24 (400 mg, 1.04 mmol) and Et₃N (0.57 mL, 4.16 mmol) in CH₂Cl₂ (10 mL) was stirred in an ice bath. Thionyl chloride (0.12 mL, 1.56 mmol) in CH₂Cl₂ (2 mL) was then added dropwise over 15 minutes, and the mixture was stirred for an additional 30 minutes. The mixture was poured into ice-cold water and extracted with CH₂Cl₂ (3×30 mL). The combined organic layers were washed with brine and dried over Na₂SO₄. The solvent was removed under reduced pressure and the residue was dried under high vacuum for 1 hour. The diasteromeric mixture of cyclic sulfites was dissolved in a mixture of CH₃CN:CCl₄ (1:1, 25 mL) and sodium periodate (333 mg, 1.56 mmol) and RuCl₃ (10 mg) were added, followed by water (2 mL). The reaction mixture was stirred for 2 hours at room temperature, then filtered through a bed of Celite, and washed with ethyl acetate. The volatile solvents were removed, and the aqueous solution was extracted with EtOAc (2×30 mL). The combined organic layers were washed with brine, dried over Na₂SO₄, concentrated under reduced pressure, and the residue purified by flash column chromatography (EtOAc/Hexanes (1:2)) to give 16 as a colorless syrup (325 mg, 70%). [α]_{D}²³=+1.2 (c=0.85, CH₂Cl₂). ¹H NMR (CDCl₃) δ 4.77-4.65 (6H, m, 3CH₂OMe), 4.62 (1H, t, J_{2,3}=J_{4,3}=9.1 Hz, H-3), 4.54 (1H, t, J_{1a,1b}=J_{2,1a}=11.1 Hz, H-1a), 4.37 (1H, dd, J_{2,1a}=5.4, J_{1a,1b}=11.1 Hz, H-1b), 4.16 (2H, m, H-2, H-6), 3.98 (1H, d, J_{4,5}=9.8 Hz, H-5), 3.80 (1H, dd, J_{6,7a}=4.8, J_{7a,7b}=10.8 Hz, H-7a), 3.75 (1H, t, J_{3,4}=J_{4,5}=8.6 Hz, H-4), 3.64 (1H, J_{7a,7b}=J_{6,7b}=8.9 Hz, H-7b), 3.44, 3.41, 3.39 (9H, 3s, 3OMe), 1.38, 1.36 (6H, 2s, 2Me).¹³C NMR (CDCl₃) δ 102.3 (CMe₂), 97.9, 96.7, 96.1 (3CH₂OMe), 89.2 (C-3), 76.9 (C-4), 74.6 (C-6), 72.2 (C-1), 71.0 (C-5), 66.8 (C-5), 66.8 (C-7), 56.5 (C-2), 56.6, 55.7, 55.3 (3OMe), 24.4, 23.9 (2Me). HRMS Calcd for C₁₆H₃₁O₁₂S (M+H): 447.1531. Found: 447.1516.

### 3,6-O-isopropylidene-1,2,4-tri-O-methoxymethyl-D-glycero-D-galacto-heptitol-5,7-cyclic sulfate (26)

Compound 26 was obtained as a colorless syrup (220 mg, 76%) from 25 (250 mg, 0.65 mmol) using the same procedure that was used to obtain 16. [α]_{D}²³=−32 (c=0.46, CH₂Cl₂). ¹H NMR (CDCl₃) δ 4.83-4.63 (6H, m, 3CH₂OMe), 4.70 (1H, m, H-5), 4.55 (1H, t, J_{7a,7b}=J_{6,7a}=11.1 Hz, H-7a), 4.39 (1H, dd, J_{6,7a}=4.9, J_{7a,7b}=10.7 Hz, H-7b), 4.24 (1H, td, J_{5,6}=5.7, J_{6,7}=10.5 Hz, H-6), 4.09 (1H, ddd, J_{1a,2}=6.9, J_{1b,2}=5.3, J_{3,2}=1.4 Hz, H-2), 3.97 (1H, dd, J_{4,3}=10.0, J_{3,2}=1.5 Hz, H-3), 3.89 (1H, dd, J_{3,4}=10.0, J_{5,4}=7.7 Hz, H-4), 3.80 (1H, dd, J_{2,1a}=5.4, J_{1a,1b}=9.8 Hz, H-1a), 3.58 (1H, t, J_{1a,1b}=J_{2,1b}=9.2 Hz, H-1b), 3.45, 3.41, 3.39 (9H, 3s, 3OMe), 1.43, 1.37 (6H, 2s, 2Me).¹³C NMR (CDCl₃) δ 102.32 (CMe₂), 98.1, 98.0, 97.9 (3CH₂OMe), 89.6 (C-5), 76.6 (C-4), 75.1 (C-2), 72.0 (C-7), 68.9 (C-3), 66.2 (C-1), 59.5 (C-6), 56.4, 56.0, 55.7 (3OMe), 24.8, 23.8 (2Me). HRMS Calcd for C₁₆H₃₀NaO₁₂S (M+Na): 470.1383. Found: 470.1399.

### 2,3,5-Tri-O-p-methoxybenzyl-1,4-dideoxy-1,4-[[2S,3S,4R,5R,6R]-2,5-isopropylidene-4,6,7-tri-O-methoxymethyl-3-(sulfooxy)heptyl]-(R)-epi-sulfoniumylidine-D-arabinitol Inner Salt (27)

The cyclic sulfate 16 (260 mg, 0.58 mmol) and the thiosugar 7 (360 mg, 0.70 mmol) were dissolved in HFIP (1.5 mL), containing anhydrous K₂CO₃ (10 mg). The mixture was stirred in a sealed reaction vessel in an oil bath at 72° C. for 6 days. The progress of the reaction was followed by TLC analysis (developing solvent EtOAc:MeOH, 10:1). The mixture was cooled, then diluted with EtOAc and evaporated to give a syrupy residue. Purification by column chromatograghy (EtOAc/MeOH 99:1) gave the sulfonium salt 27 as a syrup (360 mg, 65%). [α]_{D}²³=+62 (c=0.85, CH₂Cl₂). ¹H NMR (acetone-d₆) δ 7.32-6.91 (12H, m, Ar), 5.12-4.52 (12H, m, 3CH₂OMe, 3CH₂-Ph), 4.69 (1H, m, H-2), 4.55 (1H, m, H-3), 4.39-4.30 (4H, m, H-1′a, H-2′, H-3′, H-6′), 4.08 (1-H, t, J_{3,4}=J_{5,4}=7.4 Hz, H-4), 4.02-3.90 (4H, m, H-1a, H-1′b, H-5′), 3.85-3.78 (3H, m, H-5a, H-7′a, H-1b), 3.82 (9H, s, 3Ph-OMe), 3.60 (1H, t, J_{7′a,7′b}=J_{6′,7′b}=9.1 Hz, H-7′b), 3.42 (1H, m, H-4′), 3.39, 3.36, 3.33 (9H, 3s, 3CH₂OMe) 1.37, 1.32 (611, 2s, 2Me).¹³C NMR (acetone-d₆) δ 159.8-129 (m, Ar), 101.6 (CMe₂), 98.7, 96.5, 95.2 (3CH₂OMe), 83.5 (C-3), 81.2 (C-2), 79.7 (C-2′), 78.6 (C-4′), 74.0 (C-6′), 72.7, 71.6, 71.4 (3CH₂Ph), 71.3 (C-5′), 66.9 (C-7′), 66.6 (C-3′), 66.5 (C-5), 65.1 (C-4), 55.9-54.2 (6OMe), 51.5 (C-1′), 47.4 (C-1), 24.4, 23.5 (2Me). HRMS Calcd for C₄₅H₆₅O₁₈S₂ (M+H): 957.3607. Found: 957.3604.

### 1,4-Dideoxy-1,4[[2S,3S,4R,5R,6R]-2,4,5,6,7-pentahydroxy-3-(sulfooxy)heptyl]-(R)-epi-sulfoniumylidine]-D-arabinitol Inner Salt (6)

The protected sulfonium salt 27 (150 mg, 0.16 mmol) was dissolved in 30% aqueous solution of TFA (25 mL) and the mixture was stirred at 50° C. for 5 hours. The solvent was removed under reduced pressure and the residue was dissolved in water (5 mL) and washed with CH₂Cl₂ (3×5 mL). The water layer was evaporated to give the crude product that was purified on silica gel with EtOAc/MeOH/H₂O 6:3:1 (v/v) as eluent to give compound 6 as a colorless solid (61 mg, 93%). Mp 82-84° C. [α]_{D}²³=+5.5° (c=0.55, CH₂Cl₂). ¹H NMR (D₂O) δ 4.67 (1H, dd, J_{1a,2}=3.7, J_{1b,2}=7.4 Hz, H-2), 4.56 (1H, d, J_{2′,3′}=8.2 Hz, H-3′), 4.39 (1H, t, J_{2,3}=J_{3,4}=3.1 Hz, H-3), 4.35 (1H, dt, J_{2′,3′}=3.3, J_{2′,1′}=7.8 Hz, H-2′), 4.02 (3H, m, H-5a, H-1′a, H-4), 3.91-3.83 (5H, m, H-6′, H-5′, H-5b, H-4′, H-1′b), 3.81 (2H, d, J_{1,2}=3.9 Hz, H-1a,b), 3.71 (1H, dd, J_{7′a,7′b}=3.2, J_{7′b,6′}=11.9 Hz, H-7′b), 3.62 (1H, dd, J_{7′b,7′a}=7.8, J_{7′a,6′}=11.6 Hz, H-7′a). ¹³C NMR (D₂O) δ 78.3 (C-3′), 77.7 (C-3), 76.7 (C-2), 72.9 (C-6′), 70.7 (C-5′), 70.0 (C-4), 69.1 (C-4′), 66.0 (C-2′), 61.6 (C-7′), 59.2 (C-5), 50.7 (C-1′), 47.7 (C-1). HRMS Calcd for C₁₂H₂₅O₁₂S₂ (M+H): 425.0782. Found: 425.0778.

### 1,4-Dideoxy-1,4[[2S,3S,4R,5R,6S]-2,4,5,6,7-pentahydroxy-3-(sulfooxy)heptyl]-(R-)epi-sulfoniumylidine]-D-arabinitol Inner Salt (4)

A mixture of the thiosugar 7 (100 mg, 0.224 mmol) and the cyclic sulfate 26 (137 mg, 0.269 mmol) in HFIP (1 mL) containing K₂CO₃ (5 mg) was placed in a sealed reaction vessel and heated at 72° C. with stirring for 6 days. The progress of the reaction was followed by TLC analysis (developing solvent EtOAc:MeOH, 10:1). The mixture was cooled, then diluted with EtOAc and evaporated to give a syrupy residue. Purification by column chromatograghy (EtOAc/MeOH 95:5) gave the protected sulfonium salt as a foam (120 mg, 57%). The protected sulfonium salt 28 (100 mg, 0.11 mmol) was dissolved in 30% aqueous TFA (10 mL) and stirred at 50° C. for 5 hours. The solvents were removed under reduced pressure and the residue was dissolved in water (5 mL) and washed with CH₂Cl₂ (3×5 mL). The water layer was evaporated to give the crude product that was purified on silica gel column with EtOAc/MeOH/H₂O 6:3:1 (v/v) as eluent to give compound 4 as a colorless solid (40 mg, 91%).¹²

### Example 2.0

### Nitrogen and Selenium Analogues of Kotalanol (32, 34) and de-O-sulfonated kotalanol (33, 35)

General Methods.

Optical rotations were measured at 23° C. and reported in deg dm⁻¹ g⁻¹ cm³. ¹H and ¹³C NMR spectra were recorded at 600 and 150 MHz, respectively. All assignments were confirmed with the aid of two-dimensional ¹H, ¹H(COSY) and/or ¹H, ¹³C(HSQC) experiments using standard pulse programs. Processing of the spectra was performed with MestRec and/or MestReNova software. Analytical thin-layer chromatography (TLC) was performed on aluminum plates precoated with silica gel 60E-254 as the adsorbent. The developed plates were air-dried, exposed to UV light and/or sprayed with a solution containing 1% Ce(SO₄)₂ and 1.5% molybdic acid in 10% aqueous H₂SO₄, and heated. Column chromatography was performed with Silica gel 60 (230-400 mesh). High resolution mass spectra were obtained by the electrospray ionization method, using an Agilent 6210 TOF LC/MS high resolution magnetic sector mass spectrometer.

Enzyme Kinetics.

Activity of recombinant N-terminal domain of Maltase-Glucoamylase (ntMGAM) was determined using the glucose oxidase assay²² to follow the production of glucose from maltose upon addition of the enzyme (0.8 nM). A no-inhibitor control and five different inhibitor concentrations were used in combination with 7 different maltose concentrations (ranging from 1.5 to 24 mM). A reaction time of 60 minutes at 37° C. was employed. Reactions were linear within this time frame. Values of Kᵢ and standard deviations were determined by the program GraFit 4.0.14 (Erithacus Software)²² which employs nonlinear fitting of the data for each inhibitor concentration to the Michaelis-Menten equation.

### 7′-[(1,4-Dideoxy-1,4-imino-D-arabinitol)-4-N-ammonium]-7′-deoxy-D-perseitol-5′-sulfate (32)

The cyclic sulfate 42¹² (526 mg, 0.73 mmol) and the iminoarabinitol 40²⁵ (300 mg, 0.61 mmol) were dissolved in acetone (3 mL), and anhydrous K₂CO₃ (20 mg) was added. The mixture was stirred in a sealed tube in an oil bath at 60° C. for 5 days. The solvent was removed under reduced pressure, and the product was purified through a short silica column with EtOAc/MeOH (95:5) as eluent to yield the protected ammonium salt (43, 503 mg, 82% yield based on 50 mg recovery of unreacted iminoarabinitol 40). However, the coupled product 43 was unstable, probably due to partial deprotection of the PMB protecting groups, as indicated by TLC. Hence, without any further characterization, to a solution of the protected compound 43 (400 mg, 0.33 mmol) in CH₂Cl₂ (0.5 mL) was added trifluoroacetic acid (10 mL), followed by H₂O (1.0 mL), and the mixture was stirred at room temperature for 3 hours. The solvents were then evaporated under reduced pressure, and the residue was dissolved in water (5 mL) and washed with CH₂Cl₂ (3×5 mL). The water layer was evaporated to give a crude product that was purified on silica gel column with tOAc/MeOH/H₂O (7:3:1) (v/v) as eluent to give compound 32 as a colorless foam (108 mg, 80%). [α]_{D}²³=+6.4 (c=1.4, H₂O). ¹H NMR (D₂O, pH=8 by adding K₂CO₃): δ 4.62 (1H, d, J_{2′,3′}=4.8 Hz, H-3′), 4.06-4.03 (2H, m, H-2′, H-2), 3.88 (1H, td, J_{5′,6′}=1.2, J_{6′,7′a}=J_{6′,7′b}=6.0 Hz, H-6′), 3.85-3.83 (2H, m, H-3, H-4′), 3.66 (1H, dd, J_{4′,5′}=9.6 Hz, H-5′), 3.65-3.59 (4H, m, H-5a, H-5b, H-7′a, H-7′b), 3.21 (1H, dd, J_{1′a,2′}=6.6, J_{1′a,1′b}=12.6 Hz, H-1′a), 3.06 (1H, br d, J_{1a,1b}=11.4 Hz, H-1a), 2.78 (1H, dd, J_{1b,2}=5.4 Hz, H-1b), 2.52 (1H, q, J=4.8 Hz, H-4), 2.45 (1H, dd, J_{1′b,2′}=6.6 Hz, H-1′b). ¹³C NMR (D₂O, pH=8 by adding K₂CO₃): δ 79.2 (C-3′), 78.5 (C-3), 75.6 (C-2), 72.3 (C-4), 70.7 (C-2′), 69.8 (C-6′), 68.8 (C-4′, C-5′), 63.2 (C-7′), 60.6 (C-5), 59.4 (C-1), 56.6 (C-1′). HRMS Calcd for C₁₂H₂₆NO₁₂S (M+H): 408.1175. Found: 408.1170.

### 7′-[(1,4-Dideoxy-1,4-imino-D-arabinitol)-4-N-ammonium]-7′-deoxy-D-perseitol chloride (33)

Compound 32 (26 mg, 0.06 mmol) was stirred in 5% methanolic HCl (3 mL) at room temperature for 3.5 hours. The solvent was evaporated and the residue was treated with Amberlyst A-26 resin (20 mg, chloride form) in MeOH (1 mL). After stirring for 2.5 h, the resin was removed by filtration and the solvent was evaporated to give compound 33 as a colorless syrup in quantitative yield (21 mg). [α]_{D}²³=+6.6 (c=0.75, H₂O). ¹H NMR (D₂O, pH=8 by adding K₂CO₃): δ 4.14 (1H, dt, J_{2,1b}=5.4 Hz, J_{2,1a}=J_{2,3}=2.4 Hz, H-2), 3.98 (1H, ddd, J_{6′,7′a}=6.0 Hz, J_{6′,7′b}=7.2 Hz, J_{6′,5′}=1.8 Hz, H-6′), 3.93 (1H, dd, J_{3,4}=4.8 Hz, J_{3,2}=2.4 Hz, H-3), 3.90-3.87 (2H, m, H-2′, H-3′), 3.83 (1H, d, J_{4′,5′}=9.6 Hz, H-4′), 3.75 (2H, br d, J_{5a,4b}=J_{5b,4}=5.4 Hz, H-5a, H-5b), 3.70 (2H, br dd, H-7′a, H-7′b), 3.65 (1H, dd, H-5′) 3.26 (1H, m, H-1′a), 3.18 (1H, br d, J_{1a,1b}=11.4 Hz, H-1a), 2.87 (1H, dd, H-1b), 2.62 (1H, ddd, H-4), 2.60 (1H, br d, J_{1′b,1′a}=12.0 Hz, H-1′b). ¹³C NMR (D₂O, pH=8 by adding K₂CO₃): δ 78.3 (C-3), 75.6 (C-2), 72.6 (C-3′), 72.3 (C-4), 70.2 (C-6′), 69.2 (C-5′), 68.8 (C-2′), 68.6 (C-4′), 63.3 (C-7′), 60.8 (C-5), 59.3 (C-1), 57.7 (C-1′). HRMS Calcd for C₁₂H₂₆NO₉ (M—Cl): 328.1607. Found: 328.1602.

### 1,4-Dideoxy-1,4-[[2S,3S,4R,5R,6S]-2,4,5,6,7-pentahydroxy-3-(sulfooxy)heptyl]-(R/S)-epi-selenoniumylidine]-D-arabinitol Inner Salt (34 and 46)

The cyclic sulfate 42¹² (712 mg, 0.99 mmol) and the selenoarabinitol 41²⁶ (502 mg, 0.90 mmol) were dissolved in HFIP (3 mL), and anhydrous K₂CO₃ (20 mg) was added. The mixture was stirred in a sealed tube in an oil bath at 75° C. for 5 days. The solvent was removed under reduced pressure, and the product was purified by filtration through a short silica column with EtOAc/MeOH (95:5) as eluent to yield the protected selenonium salts 44 (454 mg, 40%) and 45 (300 mg, 26%). To a solution of the protected compound 44 (370 mg, 0.29 mmol) in CH₂Cl, (0.5 mL) was added trifluoroacetic acid (5 mL), followed by H₂O (1.0 mL), and the mixture was stirred at room temperature for 2 hours. The solvents were then evaporated under reduced pressure, and the residue was dissolved in water (5 mL) and washed with CH₂Cl₂ (3×5 mL). The water layer was evaporated to give a crude product that was purified on silica gel with EtOAc/MeOH/H₂O (7:3:1) (v/v) as eluent to give compound 34 as a colorless foam (122 mg, 89%). Similarly, compound 46 was obtained from 45 (175 mg, 0.14 mmol) as a colorless foam (53 mg, 83%).

Data for 34:

[α]_{D}²³=+16.8 (c=1.2, H₂O). ¹H NMR (D₂O): δ 4.74 (1H, q, J=3.6 Hz, H-2), 4.57 (1H, dd, J_{3′,4′}=0.6, J_{2′,3′}=7.8 Hz, H-3′), 4.45 (1H, dd, J_{3,4}=3.0, J_{2,3}=3.6 Hz, H-3) 4.38 (1H, ddd, J_{1′a, 2′}=3.6, J_{1′b,2′}=6.6 Hz, H-2′), 4.12 (1H, ddd, J_{4,5a}=4.8, J_{4,5b}=8.4 Hz, H-4), 4.05 (1H, dd, J_{1′a,1′b}=12.6 Hz, H-1′a), 4.02 (1H, dd, J_{5a,5b}=12.6 Hz, H-5a), 3.91-3.90 (4H, m, H-1′b, H-4′, H-6′, H-5b), 3.74-3.73 (3H, m, H-1a, H-1b, H-5′), 3.62-3.60 (2H, m, H-7′a, H-7′b). ¹³C NMR (D₂O): δ 78.7 (C-3′), 78.4 (C-3), 77.5 (C-2), 69.9 (C-6′), 69.8 (C-4), 68.7 (C-5′), 68.0 (C-4′), 66.1 (C-2′), 63.2 (C-7′), 59.2 (C-5), 48.9 (C-1′), 44.8 (C-1). HRMS Calcd for C₁₂H₂₅O₁₂SSe (M+H): 473.0231. Found: 473.0229.

Data for 46:

[α]_{D}²³==+106.6 (c=0.5, H₂O). ¹H NMR (D₂O): δ 4.69 (1H, q, J=3.6 Hz, H-2), 4.57 (1H, dd, J_{3′,4′}=0.6, J_{2′,3′}=7.8 Hz, H-3′), 4.49 (1H, t, J=3.6 Hz, H-3), 4.36 (1H, td, J_{1′b,2′}=4.2, J_{1′a,2′}=7.8 Hz, H-2′), 4.20 (1H, m, H-4), 4.15 (1H, dd, J_{4,5a}=6.0, J_{5a,5b}=12.6 Hz, H-5a), 4.04 (1H, m, H-5b), 4.01 (1H, dd, H-1′a), 3.92-3.89 (2H, m, H-4′, H-6′), 3.86 (1H, dd, J_{1′a,1′b}=12.6 Hz, H-1′b), 3.78 (1H, dd, J_{1a,1b}=12.6 Hz, H-1a), 3.73 (1H, dd, J_{4′,5′}=9.6 Hz, H-5′), 3.63-3.60 (2H, m, H-7′a, H-7′b), 3.56 (1H, dd, H-1b). ¹³C NMR (D₂O): δ79.0 (C-3′), 78.4 (C-2), 78.1 (C-3), 69.9 (C-6′), 68.7 (C-5′), 68.1 (C-4′), 66.0 (C-2′), 63.9 (C-4), 63.2 (C-7′), 58.0 (C-5), 42.4 (C-1), 41.4 (C-1′). HRMS Calcd for C₁₂H₂₅O₁₂SSe (M+H): 473.0231. Found: 473.0229.

### 1,4-Dideoxy-1,4-[[2S,3S,4R,5R,6S]-2,3,4,5,6,7-hexahydroxy-heptyl]-(R/S)-epi-selenoniumylidine]-D-arabinitol chloride (35 and 47)

Compound 34 (25 mg, 0.05 mmol) was stirred in 5% methanolic HCl (3 mL) at room temperature for 3.5 h. The solvent was evaporated and the residue was treated with Amberlyst A-26 resin (20 mg, chloride form) in MeOH (1 mL). After stirring for 2 hours, the resin was removed by filtration and the solvent was evaporated to give 35 as a colorless syrup in quantitative yield (21 mg). Similarly, compound 47 (13 mg, quantitative) was obtained from 46 (15 mg, 0.03 mmol) as a colorless syrup.

Data for 35:

[α]_{D}²³=+15.0° (c=0.4, H₂O). ¹H NMR (D₂O): δ 4.81 (1H, q, J=3.6 Hz, H-2), 4.50 (1H, t, J=3.6 Hz, H-3), 4.24 (1H, td, J_{1′a,2′}=4.2, J_{2′,3′}J_{1′b,2′}=7.8 Hz, H-2′), 4.20 (1H, ddd, J_{4,5a}=4.8, J_{4,5b}=8.4 Hz, H-4), 4.10 (1H, dd, J_{5a,5b}=12.6 Hz, H-5a), 3.97 (1H, dd, 12.0 Hz, H-1′a), 3.96 (1H, m, H-6′), 3.94 (1H, dd, H-5b), 3.89 (1H, d, H-3′), 3.86 (1H, m, H-4′), 3.84 (1H, dd, H-1′b), 3.82 (1H, dd, J_{1a,1b}=12.0 Hz, H-1a), 3.79 (1H, dd, H-1b), 3.66 (2H, d, J=6.6 Hz, H-7′a, H-7′b), 3.64 (1H, dd, J=0.6, J=9.0 Hz, H-5′). ¹³C NMR (D₂O): δ 78.2 (C-3), 77.6 (C-2), 72.0 (C-3′), 69.9 (C-6′), 69.5 (C-4), 69.1 (C-5′), 68.1 (C-4′), 67.5 (C-2′), 63.1 (C-7′), 59.3 (C-5), 48.0 (C-1′), 45.2 (C-1). HRMS Calcd for C₁₂H₂₅ClO₉SSe (M-Cl): 393.0663. Found: 393.0658.

Data for 47:

[α]_{D}²³=+96.6° (c=0.6, H₂O). ¹H NMR (D₂O): δ 4.74 (1H, q, J=4.2 Hz, H-2), 4.50 (1H, dd, J_{3,4}=3.6 Hz, H-3), 4.28-4.21 (3H, m, H-4, H-5a, H-2′), 4.07 (1H, dd, J_{4,5b}=11.4, J_{5a,5b}=13.8 Hz, H-5b), 3.98 (1H, dd, J_{1′a,2′}=4.2, J_{1′a,1′b}=12.0 Hz, H-1′a), 3.95 (1H, td, J_{5′,6′}=1.2, J_{6′,7′a}=J_{6′,7′b}=6.0 Hz, H-6′), 3.91 (1H, dd, J_{2′,3′}=7.8, J_{4′,3′}=0.6 Hz, H-3′), 3.88-3.85 (2H, m, H-4′, H-1a), 3.84 (1H, dd, J_{2′,1′b}=8.4 Hz, H-1′b), 3.67 (2H, d, J=6.6 Hz, H-7′a, H-7′b), 3.64 (1H, dd, J_{4′,5′}=5.4 Hz, H-5′), 3.63 (1H, dd, J_{1b,2}=4.2, J_{1a,1b}=13.2 Hz, H-1b). ¹³C NMR (D₂O): δ 78.2 (C-2), 78.1 (C-3), 71.9 (C-3′), 69.9 (C-6′), 69.1 (C-5′), 68.0 (C-4′), 67.1 (C-2′), 63.9 (C-4), 63.1 (C-7′), 58.0 (C-5), 42.1 (C-1′), 41.0 (C-1). HRMS Calcd for C₁₂H₂₅ClO₉SSe (M-Cl): 393.0663. Found: 393.0658.

### 1,4-Dideoxy-1,4-[[2S,3S,4R,5S,6S]-2,3,4,5,6,7-hexahydroxy-heptyl]-(R)-epi-sulfnoniumylidine]-D-arabinitol chloride (38)

Compound 38 was obtained as a colorless foam (18 mg, quantitative) from compound 36,¹⁵ (22 mg, 0.06 mmol) using the same procedure as that described to obtain 33. [α]_{D}²³=+10.5° (c=0.5, H₂O). ¹H NMR (D₂O): S 4.77 (1H, q, J=3.6 Hz, H-2), 4.47 (1H, dd, J=3.6 Hz, H-3), 4.27 (1H, ddd, J_{1′a,2′}=3.0, J_{2′,3′}=7.2, J_{2′,1′b}=9.6 Hz, H-2′), 4.16 (1H, dd, J_{4,5a}=4.8, J_{5a,5b}=11.4 Hz, H-5a), 4.13 (1H, ddd, J_{4,5b}=7.2 Hz, H-4), 4.00 (1H, t, J=3.0 Hz, H-4′), 3.97 (1H, dd, H-5b), 3.95 (1H, dd, J_{1′a,1′b}=13.8 Hz, H-1′a), 3.94 (1H, dd, J_{1a,1b}=13.2 Hz, H-1a), 3.91 (1H, dd, H-1b), 3.84 (1H, dd, J_{2′,3′}=7.2 Hz, H-3′), 3.82 (1H, dd, H-1′b), 3.80 (1H, dd, J_{6′,7′a}=2.4, J_{7′a,7′b}=12.0 Hz, H-7′a), 3.76 (1H, ddd, J_{6′,7′b}=6.0, J_{5′,6′}=7.8, Hz H-6′), 3.73 (1H, dd, H-5′), 3.67 (1H, dd, H-7′b). ¹³C NMR (D₂O): δ 77.5 (C-3), 76.9 (C-2), 74.6 (C-3′), 72.5 (C-5′), 71.0 (C-6′), 69.9 (C-4), 68.1 (C-4′), 67.4 (C-2′), 62.4 (C-7′), 59.1 (C-5), 49.7 (C-1′), 48.2 (C-1). HRMS Calcd for C₁₂H₂₅O₉SCl (M-Cl): 345.1219. Found: 345.1210.

### 1,4-Dideoxy-1,4-[[2S,3S,4R,5S,6R]-2,3,4,5,6,7-hexahydroxy-heptyl]-(R)-epi-sulfnoniumylidine]-D-arabinitol chloride (39)

Compound 39 was obtained as a colorless foam (20 mg, quantitative) from compound 37¹⁵ (24 mg, 0.06 mmol) using the same procedure as that described to obtain 33. [α]_{D}²³=+8.3° (c=0.4, H₂O). ¹H NMR (D₂O): δ 4.78 (1H, q, J=3.6 Hz, H-2), 4.47 (1H, t, J=3.0 Hz, H-3), 4.29 (1H, ddd-like, H-2′), 4.16 (1H, dd, J_{4,5a}=4.8, J_{5a,5b}=11.4 Hz, H-5a), 4.13 (1H, ddd, J_{3,4}=1.8, J_{4,5b}=6.6 Hz, H-4), 3.99 (1H, dd, H-5b), 3.96 (2H, m, H-1′a, H-4′), 3.94 (1H, dd, J_{1a,1b}=13.2 Hz, H-1a), 3.90 (1H, dd, H-1b), 3.83 (1H, ddd, J_{5′,6′}=3.0, J_{6′,7′a}=4.8, J_{6′,7′b}=7.2 Hz, H-6′), 3.80 (2H, m, H-1′b, H-3′), 3.77 (1H, dd, J_{4′,5′}=6.6 Hz, H-5′), 3.72 (1H, dd, J_{7′a,7′b}=11.4 Hz, H-7′a), 3.67 (1H, dd, H-7′b). ¹³C NMR (D₂O): δ 77.5 (C-3), 76.9 (C-2), 72.7 (C-3′), 71.6 (C-5′), 70.9 (C-6′), 70.0 (C-4), 69.5 (C-4′), 67.4 (C-2′), 62.8 (C-7′), 59.2 (C-5), 50.0 (C-1′), 48.2 (C-1). HRMS Calcd for C₁₂H₂₅O₉SCl (M-Cl): 345.1219. Found: 345.1213.

### Example 3.0

### De-O-sulfonated ponkoranol (54) and its Stereoisomer (55)

General:

Optical rotations were measured at 23° C. ¹H and ¹³C NMR spectra were recorded at 600 and 150 MHz, respectively. All assignments were confirmed with the aid of two-dimensional ¹H, ¹H(COSYDFTP) or ¹H, ¹³C (INVBTP) experiments using standard pulse programs. Column chromatography was performed with Silica 60 (230-400 mesh). Reverse column chromatography was performed with Silica C-18 cartridges. High resolution mass spectra were obtained by the electrospray ionization method, using an Agilent 6210 TOF LC/MS high resolution magnetic sector mass spectrometer.

### Benzyl 6-deoxy-6-[2,3,5-tri-O-benzyl-1,4-dideoxy-episulfoniummylidene-D-arabinitol]-β-D-glycopyranoside-p-toluenesulfonate (62)

Benzyl 6-O-p-toluene-sulfonyl-β-D-glucopyranoside 60^{35, 36} (470 mg, 1.11 mmol) and the thioether 51^{11(a)} (558 mg, 1.33 mmol) were dissolved in HFIP (1.5 mL), containing anhydrous K₂CO₃ (10 mg). The mixture was stirred in a sealed reaction vessel in an oil bath at 70° C. for 4 days. The mixture was cooled, then diluted with EtOAc, and evaporated to give a syrupy residue. Purification by column chromatography (EtOAc/MeOH 92:8) gave the sulfonium salt 62 as a syrup (388 mg, 52%). [α]_{D}²³=+16 (c=0.8, MeOH). ¹H NMR (MeOD) δ 7.67-7.17 (24H, m, Ar), 4.87 (1H, d, J_{1′,2′}=3.6 Hz, H-1′), 4.63 (1H, m, H-3), 4.64-4.46 (8H, m, 4CH₂-Ph), 4.41 (1H, br, H-2), 4.28 (1H, dd, J_{3,4}=5.7 J_{4,5}=9.4 Hz, H-4), 3.93 (2H, m, H-1a, H-5′), 3.81 (1H, dd, J_{6′a,5′}=3.1, J_{6′a,6′b}=13.2 Hz, H-6′a), 3.77 (1H, dd, J_{5a,4}=6.0, J_{5a,5b}=10.5 Hz, H-5a), 3.72 (1H, dd, J_{1,2}=3.6, J_{1a,1b}=13.3 Hz, H-1b), 3.67-3.62 (3H, m, H-5b, H-6b, H-3′), 3.35 (1H, dd, J_{1′,2′}=3.6, J_{2′,3′}=9.8 Hz, H-2′) 3.21 (1H, t, J_{4′,5′}==J_{3′,4′}=8.9 Hz, H-4′), 2.32 (3H, s, Me).¹³C NMR (MeOD) δ 142.2-125.6 (m, Ar), 99.3 (C-1′), 83.2 (C-3), 83.0 (C-2), 73.1, 72.0, 71.9, 70.7 (4CH₂-Ph), 73.0 (C-4′), 72.9 (C-3′), 71.7 (C-2′), 68.8 (C-5′), 66.9 (C-4), 66.5 (C-5), 49.0 (C-1), 48.2 (C-6′), 19.9 (Me). HRMS Calcd for C₃₉H₄₅O₈S (M+.): 673.2830. Found: 673.2831.

### 1,4-Dideoxy-1,4-[[2S, 3S, 4R,5S]-2,3,4,5,6-pentahydroxy-hexyl]-(R)-epi-sulfoniumylidine]-D-arabinitol chloride (54)

Compound 62 (300 mg, 0.36 mmol) was dissolved in CH₂Cl₂ (25 mL), the mixture was cooled to −78° C., and BCl₃ (1M solution in CH₂Cl₂, 3.56 mmol) was added under N₂. The reaction mixture was stirred at the same temperature for 30 minutes, and then allowed to warm to 5° C. for 6 hours. The reaction was quenched by addition of MeOH (5 mL), the solvents were removed, and the residue was co-evaporated with MeOH (2×5 mL). The crude residue was dissolved in H₂O (10 mL), Amberlyst A-26 resin (200 mg) was added, and the reaction mixture was stirred at room temperature for 3 hours. Filtration through cotton, followed by solvent removal gave the crude hemiacetal. The crude product was dissolved in water (8 mL), and the solution was stirred at room temperature while NaBH₄ (67 mg, 1.78 mmol) was added in small portions over 30 minutes. Stirring was continued for another 3 hours and the mixture was acidified to pH<4 by dropwise addition of 2M HCl. The mixture was evaporated to dryness and the residue was co-evaporated with anhydrous MeOH (3×30 mL). Treatment of the solid residue with 50% EtOAc:MeOH (5-10 mL) resulted in precipitation of most of the borate salt. Filtration through cotton, followed by solvent removal gave the crude compound. The residue was purified by reverse phase column chromatography (MeOH/H₂O (2:100)) to give 54 as a colorless solid (60 mg, 48%). [α]_{D}²³=+4°. (c=0.5, H₂O). ¹H NMR (D₂O) δ 4.64 (1H, m, H-2), 4.35 (1H, t, br, H-3), 4.14 (1H, td, J_{1′,2′}=9.1, J_{2′,3′}=3.0 Hz, H-2′), 4.02 (2H, m, H-5a, H-4), 3.87-3.77 (4H, m, H-5b, H-1′a, H-1a, H-1b), 3.72-3.66 (3H, m, H-4′, H-5′, H-1′b), 3.62 (2H, m, H-6′a, H-3′), 3.50 (1H, dd, J_{6′a,6.b}=11.7, J_{5′,6′b}=5.6 Hz, H-6′b). ¹³C NMR (D₂O) δ 77.5 (C-3), 76.9 (C-2), 73.1 (C-3′), 72.7 (C-5′), 70.0 (C-4), 69.3 (C-4′), 67.4 (C-2′), 62.2 (C-6′), 59.2 (C-5), 50.0 (C-1′), 48.2 (C-1). HRMS Calcd for C₁₁H₂₃O₈S (M+.): 315.1108. Found: 315.1117.

### Benzyl 6-deoxy-6-[2,3,5-tri-O-benzyl-1,4-dideoxy-episulfoniummylidene-D-arabinitol]-β-D-mannopyranoside-p-toluenesulfonate (63)

Reaction of the thioether 51 ^{11(a)} (590 mg, 1.41 mmol) with benzyl 6-O-p-toluenesulfonyl-β-D-mannopyranoside 61³⁵ (500 mg, 1.18 mmol) in HFIP (1.5 mL), containing anhydrous K₂CO₃ (10 mg) at 70° C. for 4 days gave the sulfonium salt 63 as a foam (370 mg, 47%) after purification by column chromatography (EtOAc/MeOH (92:8)). [α]_{D}²³=+8°, (c=0.5, MeOH). ¹H NMR (MeOD) δ 7.73-7.23 (24H, m, Ar), 4.87 (1H, m, H-1′), 4.70 (1H, m, 11-2), 4.69-4.52 (8H, m, 4CH₂-Ph), 4.49 (1H, m, H-3), 4.31 (1H, t, J_{3A}=J_{4,5}=9.6 Hz, H-4), 4.04 (1H, d, br, J_{1,2}=13.1 Hz, H-1a), 3.94 (2H, m, H-6′a, H-4′), 3.90-3.85 (3H, m, H-2′, H-1b, H-5a), 3.79-3.73 (3H, m, H-6′b, H-5b, H-3′), 3.61 (1H, t, J_{4′,5′}=J_{5′,6′}=9.3 Hz, H-5′), 2.38 (3H, s, Me). ¹³C NMR (MeOD) δ 142.2-125.6 (m, Ar), 100.5 (C-1′), 83.3 (C-2), 82.9 (C-3), 73.1, 72.0, 71.8, 70.1 (4CH₂-Ph), 70.6 (C-2′), 70.4 (C-3′), 66.7 (C-4), 66.5 (C-5), 48.8 (C-1), 48.2 (C-6′), 19.9 (Me). HRMS Calcd for C₃₉H₄₅O₈S (M+.): 673.2830. Found: 673.2828.

### 1,4-Dideoxy-1,4-[[2S, 3S, 4R,5R]-2,3,4,5,6-pentahydroxy-hexyl]-(R)-epi-sulfoniurnylidine]-D-arabinitol chloride (55)

Compound 55 was obtained as a colorless solid (51 mg, 41%) from 63 (300 mg, 0.36 mmol) using the same procedure that was used to obtain 54. [α]_{D}²³=+11°, (c=0.3, H₂O). ¹H NMR (D₂O) δ 4.65 (1H, d, br, 4.35 (1H, t, br, H-3), 4.12 (1H, td, J_{1′,2′}=9.1, J_{2′,3′}=3.0 Hz, H-2′), 4.02 (2H, m, H-5a, H-4), 3.89-3.60 (9H, m, H-1′a, H-5b, H-1a, H-1b, H-3′, H-6′a, H-1′b, H-4′, H-5′), 3.55 (1H, dd, J_{6′a,6,b}=11.7, J_{5′,6′b}=5.8 Hz, H-6′b). ¹³C NMR (D₂O) δ 77.5 (C-3), 76.9 (C-2), 71.5 (C-3′), 70.5 (C-5′), 70.0 (C-4), 68.8 (C-4′), 67.3 (C-2′), 63.0 (C-6′), 59.2 (C-5), 50.4 (C-1′), 48.1 (C-1). HRMS Calcd for C₁₁H₂₃O₈S (M+.): 315.1108. Found: 315.1122.

### Example 4.0

### Selenium Analogue of C-5′ Epimer of de-O-sulfonated ponkoranol (66)

### Benzyl 6-deoxy-6-[2,3,5-tri-O-benzyl-1,4-dideoxy-(R)-epi-seleniumylidene-D-arabinitol]-α-D-mannopyranoside-p-toluenesulfonate (65)

Reaction of the 1,4-dideoxy-2,3,5-tri-O-benzyl-1,4-anhydro-4-seleno-D-arabinitol 64²⁰ (660 mg, 1.4 mmol) with benzyl 6-O-p-toluenesulfonyl-β-D-mannopyranoside 61 (500 mg, 1.2 mmol) in HFIP (1.5 mL), containing anhydrous K₂CO₃ (10 mg) at 65-70° C. for 4 days gave the selenonium salt 65 as a foam (473 mg, 45%) after purification by column chromatography (CHCl₃/MeOH (95:5)). ¹H NMR (MeOD) δ 7.74-7.24 (24H, m, Ar), 4.86 (1H, m, H-1′), 4.81 (1H, m, H-2), 4.71-4.50 (8H, m, 4CH₂-Ph), 4.58 (1H, m, H-3), 4.42 (1H, dd, J_{3,4}=6.8, J_{4,5}=9.4 Hz, H-4), 4.03 (1H, d, J=_{1a,1b}=J_{1,2}=12.8 Hz, H-1a), 3.94 (2H, m, H-6′a, H-4′), 3.88 (1H, dd, J_{1′,2′}=2.0 J_{2′,3′}=2.7 Hz, H-2′) 3.83 (1H, dd, J_{4,5}=6.7, J_{5a,5b}=10.3 Hz, H-5a), 3.78-3.73 (4H, m, H-1b, H-5b, H-3′, H-6b), 3.59 (1H, t, J_{4′,5′}=J_{5′,6′}=9.3 Hz, H-5′), 2.39 (3H, s, Me).¹³C NMR (MeOD) 141.7-125.1 (m, Ar), 100.0 (C-1′), 83.7 (C-2), 83.2 (C-3), 72.6, 71.5, 71.2, 69.5 (4CH₂-Ph), 70.2 (C-2′), 70.0 (C-5′), 69.9 (C-3′), 68.9 (C-4′), 66.1 (C-5),65.7 (C-4), 45.9 (C-1), 45.6 (C-6′), 19.4 (Me). HRMS Calcd for C₃₉H₄₅O₈Se (M+.): 721.2278. Found: 721.2278.

### 1,4-Dideoxy-1,4-[[2S, 3S, 4R,5R]-2,3,4,5,6-pentahydroxy-hexyl]-(R)-epi-seleniumylidenel-D-arabinitol chloride (66)

Compound 65 (200 mg, 0.22 mmol) was dissolved in CH₂Cl₂ (20 mL), the mixture was cooled to −78° C., and BCl₃ (1M solution in CH₂Cl₂, 3.6 mmol) was added under N₂. The reaction mixture was stirred at the same temperature for 30 minutes, and then allowed to warm to −5° C. for 6 hours. The reaction was cooled to −78° C. and quenched by addition of MeOH (5 mL), the solvents were removed, and the residue was co-evaporated with MeOH (2×5 mL). The crude residue was dissolved in H₂O (10 mL), Amberlyst A-26 resin (200 mg) was added, and the reaction mixture was stirred at room temperature for 3 hours. Filtration through cotton, followed by solvent removal gave the crude hemiacetal. The crude product was dissolved in water (8 mL), and the solution was stirred at room temperature while NaBH₄ (34 mg, 0.9 mmol) was added in small portions over 30 minutes. Stirring was continued for another 3 hours and the mixture was acidified to pH<4 by dropwise addition of 2M HCl. The mixture was evaporated to dryness and the residue was co-evaporated with anhydrous MeOH (3×30 mL). Treatment of the solid residue with 50% EtOAc:MeOH (5-10 mL) resulted in precipitation of most of the borate salt. Filtration through cotton, followed by solvent removal gave the crude compound 66. ¹H NMR (D₂O) δ 4.76 (1H, d, br, H-2), 4.45 (1H, t, J_{3,4}=J_{2,3}=3.3 Hz, H-3), 4.18 (2H, m, H-2′, H-4), 4.07-3.67 (10H, m, H-5a, H-1′a, H-5b, H-1a, H-1b, H-3′, H-6′a, H-1′b, H-4′, H-5′), 3.60 (1H, dd, J_{6′a,6.b}=13.7, J_{5′,6′b}=5.3 Hz, H-6′b). ¹³C NMR (D₂O).δ 78.3 (C-3), 77.9 (C-2), 72.1 (C-3′), 71.1 (C-4), 70.7 (C-5′), 69.8 (C-4′), 67.5 (C-2′), 63.0 (C-6′), 59.4 (C-5), 47.9 (C-1′), 46.1 (C-1). HRMS Calcd for C₁₁H₂₃O₈Se (M+.): 363.0553. Found: 363.0544.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true scope.

### REFERENCES

1) Dwek, R. A. Chem. Rev. 1996, 96, 683-720.
2) Varki, A.; Cummings, R.; Esko, J.; Freeze, H.; Hart, G.; Marth, J. Essentials of Glycobiology; Cold Spring Harbor Laboratory Press: Cold Spring Harbor, N.Y., 1999.
3) For a recent review on glycosidase inhibitors: de Melo, E. B.; Gomes, A. D.; Carvalho, I. Tetrahedron 2006, 62, 10277-10302.
4) Chandrasena, J. P. C. The Chemistry and Pharmacology of Ceylon and Indian medicinal plants, H&C Press, Colombo, Sri Lanka, 1935.
5) Jayaweera, D. M. A. Medicinal Plants Used in Ceylon-Part 1, National Science Council of Sri Lanka: Colombo, 1981, p. 77.
6) Vaidyartanam, P. S. In Indian Medicinal Plants: a compendium of 500 species, Warner, P. K.; Nambiar, V. P. K.; Ramankutty, C.; Eds. Orient londman: India 1993, pp. 47-48.
7) Yoshikawa, M.; Murakami, T.; Shimada, H.; Matsuda, H.; Yomahara, J.; Tanabe, G.; Muraoka, O. Tetrahedron Lett. 1997, 38, 8367-8370.
8) Yoshikawa, M.; Murakami, T.; Yashiro, K.; Matsuda, H.; Chem. Pharm. Bull. 1998, 46, 1339-1340.
9) Yoshikawa, M.; Xu, F.; Nakamura, S.; Wang, T.; Matsuda, H.; Tanabe, G.; Muraoka, O. Heterocycles 2008, 75, 1397-1405.
10) Muraoka, O.; Xie, W.; Tanabe, G.; Amer, M. F. A.; Minematsu, T.; Yoshikawa, M. Tetrahedron Lett. 2008, 49, 7315-7317.
11) For reviews see: a) Mohan, S.; Pinto, B. M. Carbohydr. Res. 2007, 342, 1551-1580. b) Mohan, S.; Pinto, B. M. Collect. Czech. Chem. Commun. 2009, 74, 1117-1136.
12) Jayakanthan, K.; Mohan, S.; Pinto, B. M. J. Am. Chem. Soc. 2009, 131, 5621-5626.
13) Baggett, N.; Stribblehill, P. J. Chem. Soc., Perkin Trans. 1, 1977, 1123-1126.
14) Harris, J. M.; Keranen, M. D.; O'Doherty, G. A. J. Org. Chem. 1999, 64, 2982-2983.
15) Nasi, R.; Patrick, B. O.; Sim, L.; Rose, D. R.; Pinto, B. M. J. Org. Chem. 2008, 73, 6172-6181.
16) Ghavami, A.; Sadalapure, K. S.; Johnston, B. D.; Lobera, M.; Snider, B. B.; Pinto, B. M. Synlett 2003, 9, 1259-1262.
17) Sim, L.; Jayakanthan, J.; Mohan, S.; Nasi, R.; Johnston, B. D.; Pinto, B. M.; Rose, D. R. Biochemistry 2010, 49, 443-451.
18) Ghavami, A.; Johnston, B. D.; Jensen, M. T.; Svensson, B.; Pinto, B. M. J. Am. Chem. Soc. 2001, 123, 6268-6271.
19) Muraoka, O.; Ying, S.; Yoshikai, K.; Matsuura, Y.; Yamada, E.; Minematsu, T.; Tanabe, G.; Matsuda, H.; Yoshikawa, M. Chem. Pharm. Bull. 2001, 49, 1503-1505.
20) Johnston, B. D.; Ghavami, A.; Jensen, M. T.; Svensson, B.; Pinto, B. M. J. Am. Chem. Soc. 2002, 124, 8245-8250.
21) (a) Johnston, B. D.; Jensen, H. H.; Pinto, B. M. J. Org. Chem. 2006, 71, 1111-1118. (b) Nasi, R.; Sim, L.; Rose, D. R.; Pinto, B. M. J. Org. Chem. 2007, 72, 180-186.
22) Rossi, E. J.; Sim, L.; Kuntz, D. A.; Hahn, D.; Johnston, B. D.; Ghavami, A.; Szczepina, M. G.; Kumar, N. S.; Sterchi, E. E.; Nichols, B. L.; Pinto, B. M.; Rose, D. R. FEBS J. 2006, 273, 2673-2683.
23) Pinto, B. M.; Johnston, B. D.; Ghavami, A.; Szczepina, M. G.; Liu, H.; Sadalapure, K., US patent application publication No. 2005/0065139, filed Jun. 25, 2004.
24) Ozaki, S.; Oe, H.; Kitamura, S. J. Nat. Prod. 2008, 71, 981-984.
25)Liu, H.; Sim, L.; Rose, D. R.; Pinto, B. M. J. Org. Chem. 2006, 71, 3007-3013.
26)Liu, H.; Pinto, B. M. J. Org. Chem. 2005, 70, 753-755.
27) (a) Potterat, O.; Hamburger, M. In Natural Compounds as Drugs Volume I; Petersen, F.; Amstutz, R., Eds.; Progress in Drug Research; Birkhäuser Basel: 2008; vol 65, p. 45-118. (b) Farnsworth, N. R.; Akerele, O.; Bingel, A. S.; Soejarto, D. D.; Guo, Z. Bull. W. H. O. 1985, 63, 965-981. (c) Koehn, F. E.; Carter, G. T.; Nat. Rev. Drug Discov. 2005, 4, 206-220. (d) Newman, D. J.; Cragg, G. M. J. Nat. Prod. 2007, 70, 461-477. (e) Matsuda, H.; Morikawa, T.; Yoshikawa, M. Pure Appl. Chem. 2002, 74, 1301-1308.
28) Minami, Y.; Kurlyarna, C.; Ikeda, K.; Kato, A.; Takebayashi, K.; Adachi, I.; Fleet, G. W. J.; Kettawan, A.; Karnoto, T.; Asano, N. Bioorg. Med. Chem. 2008, 16, 2734-2740.
29) Tanabe, G.; Xie, W. J.; Ogawa, A.; Cao, C. N.; Minematsu, T.; Yoshikawa, M.; Muraoka, O. Bioorg. Med. Chem. Lett. 2009, 19, 2195-2198.
30) Satoh, H.; Yoshimura, Y.; Sakata, S.; Miura, S.; Machida, H.; Matsuda, A. Bioorg. Med. Chem. Lett. 1998, 8, 989-992.
31) Matsuda, H.; Yoshikawa, M.; Murakami, T.; Tanabe, G.; Muraoka, O. J. Trad. Med. 2005, 22, 145-153.
32) Skaanderup, P. R.; Poulsen, C. S.; Hyldtoft, L.; Jorgensen, M. R.; Madsen, R. Synthesis 2002, 1721-1727.
33) Sankar, M.; Sim, L.; David, R. R.; Pinto, B. M. Carbohydr. Res. 2007, 342, 901-912.
34) Andreana, P. R.; Sanders, T.; Janczuk, A;.Warrick, J. I.; Wang, P. G. Tetrahedron Lett. 2002, 43, 6525-6528.
35) Branchaud, B. P.; Meier, M. S. J. Org. Chem. 1989, 54, 1320-1326.
36) Koto, S.; Inada, S.; Yoshida, T.; Toyama, M.; Zen, S. Can. J. Chem. 1981, 59, 255-259.
37) Chehade, J. M.; Mooradian, A. G. Drugs 2000, 60(1): 95-113.
38) Rybka, J.; Goke, B.; Sissmann, J. Diabetes 1999, 48 (Suppl 1): A101.

1. A ponkoranol derivative having the structure I, II, III, IV, V, VI, VII, VIII, IX, X, XI or XII:

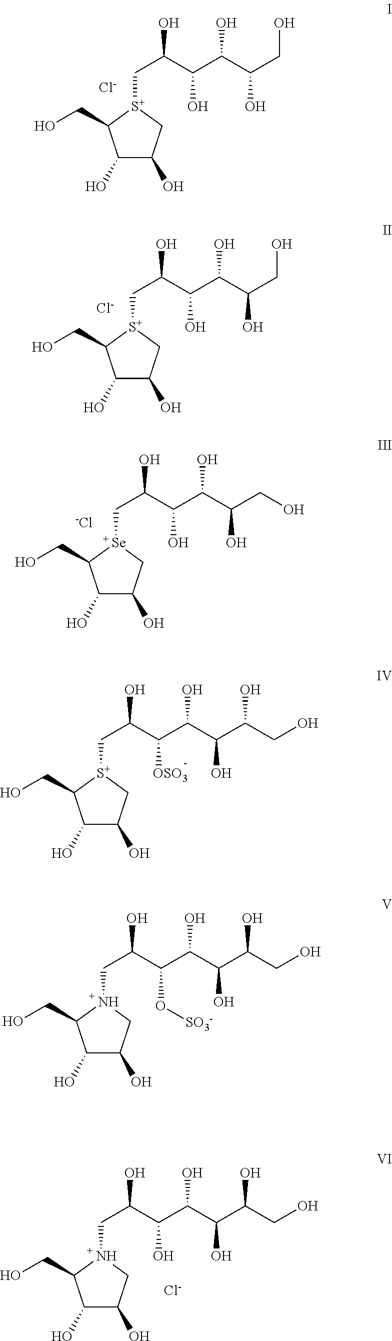

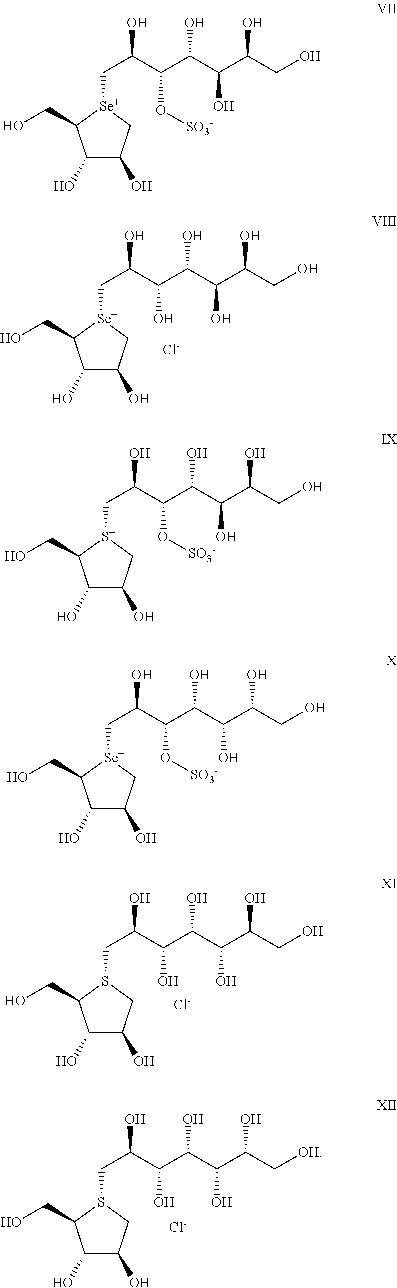

2. A method of synthesizing a compound having the structure I as defined in claim 1, the method comprising the steps set forth in Scheme I:

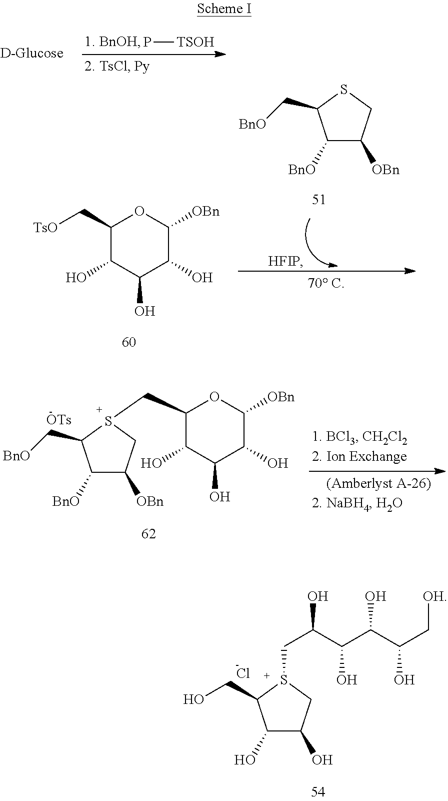

3. A method of synthesizing a compound having the structure II as defined in claim 1, the method comprising the steps set forth in Scheme II:

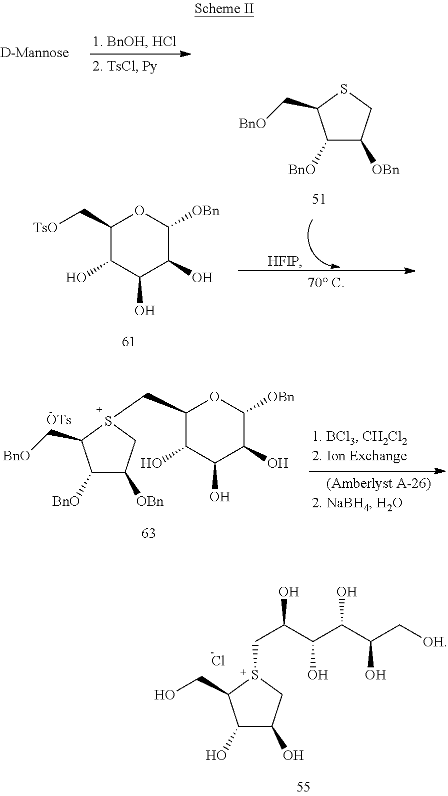

4. A method of synthesizing a compound having the structure III as defined in claim 1, the method comprising the steps set forth in Scheme III:

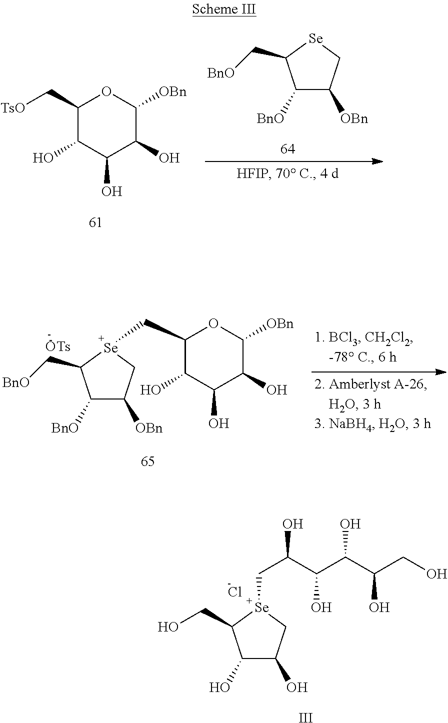

5. (canceled)

6. A method of synthesizing a compound having the structure IV as defined in claim 1, the method comprising the steps set forth in Schemes IV, V and VI:

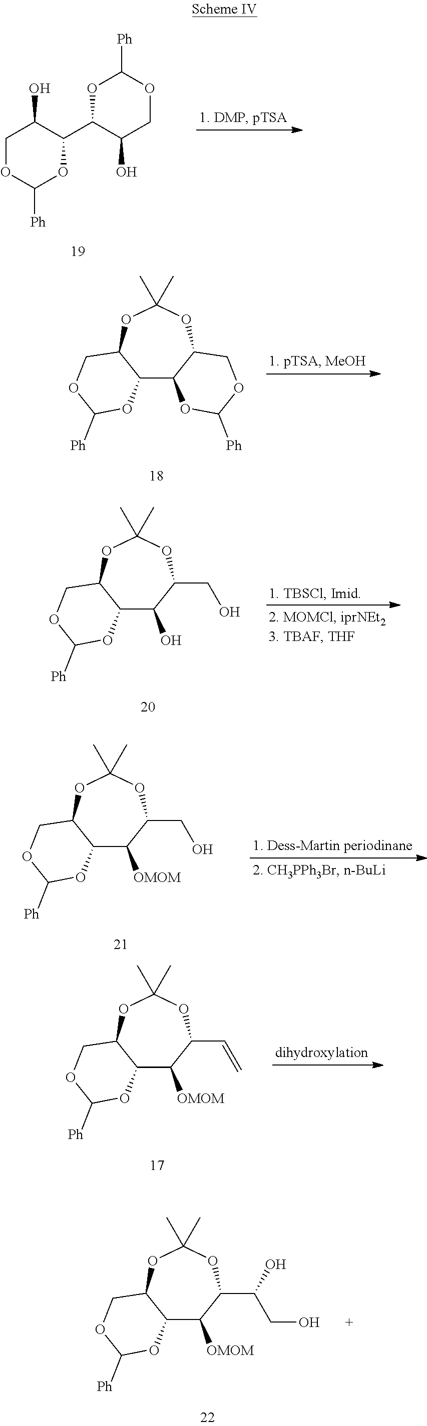

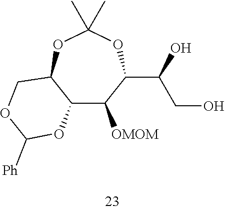

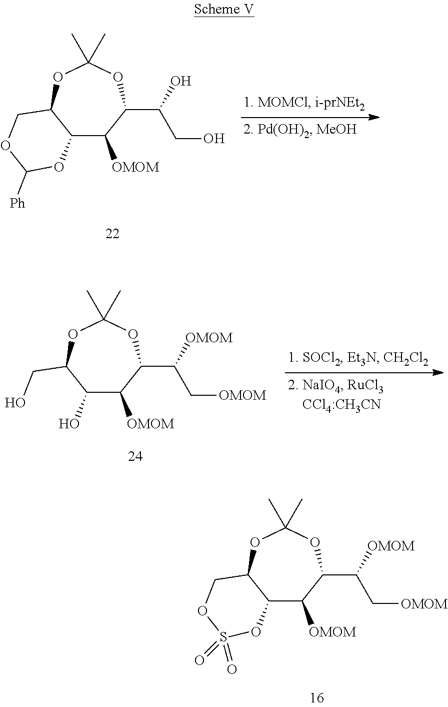

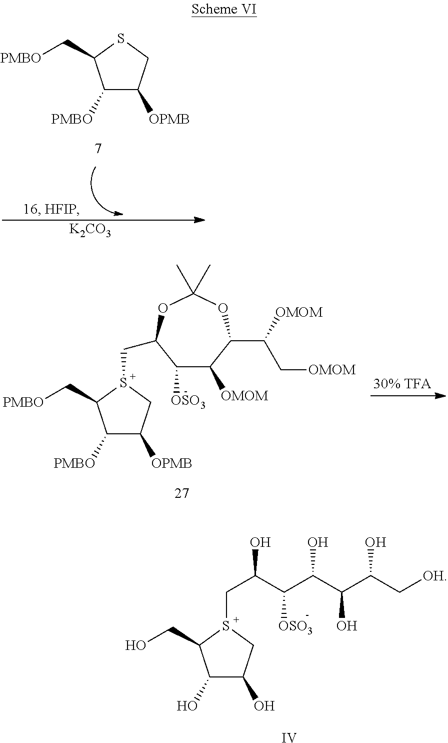

7. A method of synthesizing kotalanol, the method comprising the steps set forth in Schemes VII, VIII and IX:

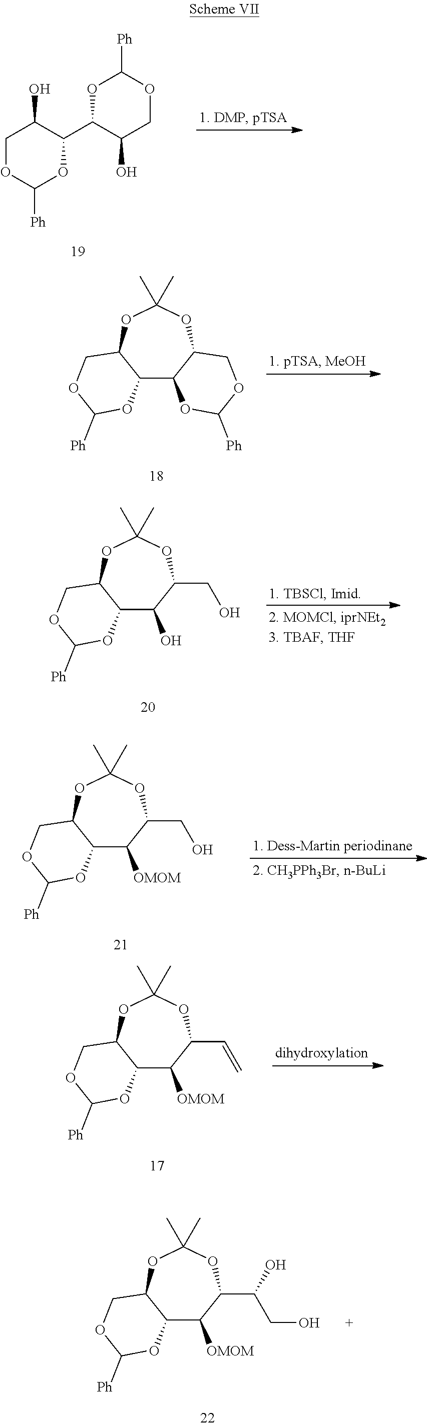

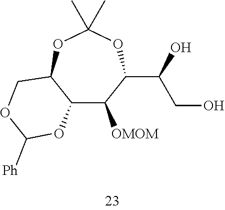

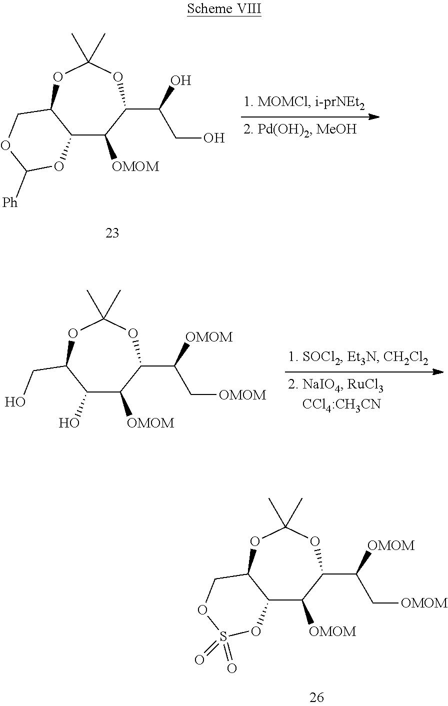

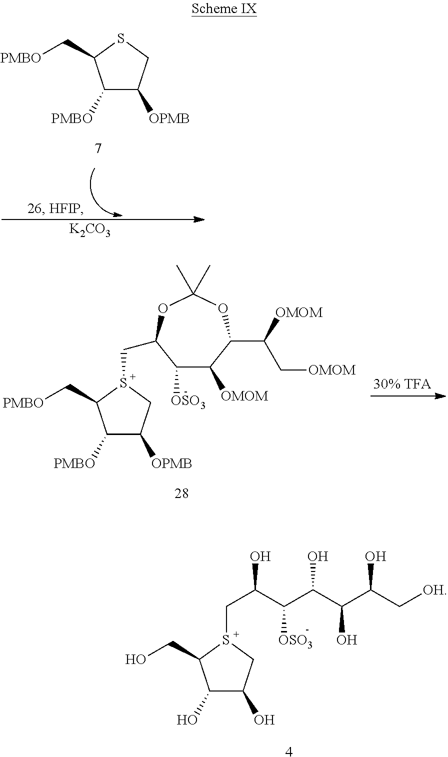

8. (canceled)

9. (canceled)

10. A method of synthesizing a compound having the structure V as defined in claim 1, the method comprising the steps set forth in Scheme X:

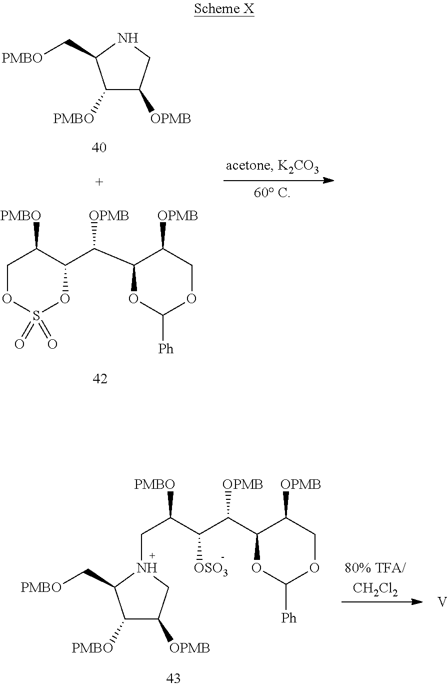

11. A method of synthesizing a compound having the structure VII as defined in claim 1, the method comprising the steps set forth in Scheme XI:

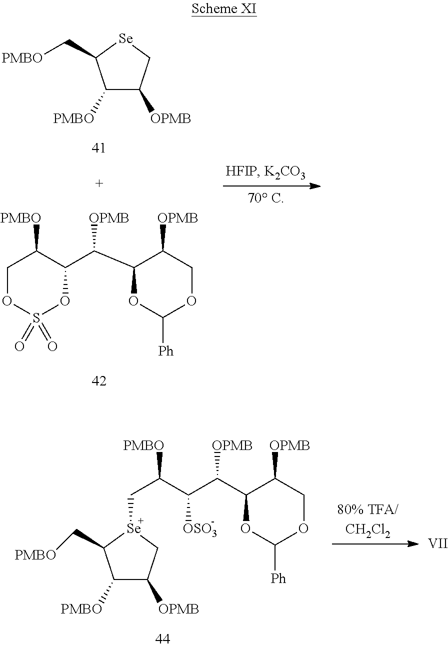

12. A method of synthesizing a compound having the structure VI, VIII, XI or XII as defined in claim 1, the method comprising the steps set forth in Scheme XII:

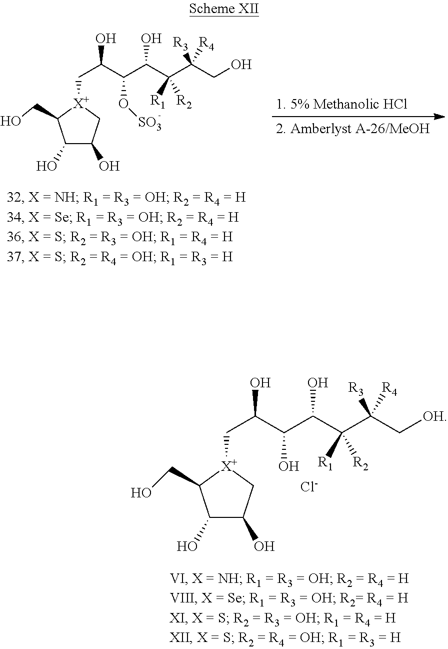

13. A method of using a compound as defined in claim 1 as a glycosidase inhibitor, comprising administering said compound to a patient.

14. A method for treating diabetes in an affected patient comprising the step of administering to the patient a therapeutically effective amount of a compound as defined in claim 1.

15. (canceled)

16. (canceled)
